SEQUENCE LISTING (11 sequences)

SEQ ID NO: 1 - DNA, 20 bp, Artificial Sequence
  P450F primer
cagaccatggaactcagcgt

SEQ ID NO: 2 - DNA, 20 bp, Artificial Sequence
  P450R primer
ggacactgaatgaccctgga

SEQ ID NO: 3 - DNA, 34 bp, Artificial Sequence
  P450FOR primer
tcatgctagcggatccaccatggaactcagcgtc

SEQ ID NO: 4 - DNA, 35 bp, Artificial Sequence
  P450REV primer
aaaatcacactctagattccctcagccccttcagc

SEQ ID NO: 5 - DNA, 81 bp, Artificial Sequence
  Lead primer
ctcagcaccctcgagaggcctgccaccatggggactgctccaaagaagaagcgtaaggtagtcgttttacaacgtcgtgac

SEQ ID NO: 6 - DNA, 20 bp, Artificial Sequence
  Complement primer
gatcggtgcgggcctcttcg

SEQ ID NO: 7 - DNA, 27 bp, Artificial Sequence
  VSAT 79 primer
catgcatctagggcggccgcactagag

SEQ ID NO: 8 - DNA, 36 bp, Artificial Sequence
  VSAT 79 primer
ggttgtggcccatggtatcatcgtgtttttcaaagg

SEQ ID NO: 9 - DNA, 41 bp, Artificial Sequence
  Synthetic linker including EcoR1 sticky end
aattccagctgagcgccggtcgctaccattaccagttggtc

SEQ ID NO: 10 - DNA, 42 bp, Artificial Sequence
  Synthetic linker including last residue of lacZ
tggtgtcaggggatcccccgggctgcagccaatatgggatcg

SEQ ID NO: 11 - DNA, 39 bp, Artificial Sequence
  Synthetic linker including first residue of \ NeoR and XmaIII stick end
ggccggagaacctgcgtgcaatccatcttgttcaatggc

(19) United States
(12) Patent Application Publication
(10) Pub. No.: US 2007/0281898 A1
(43) Pub. Date: Dec. 6, 2007

(54) VECTOR

(75) Inventors: Andrew Slade, Oxford (GB); Susan Kingsman, Oxford (GB)

Correspondence Address: FROMMER LAWRENCE & HAUG, 745 FIFTH AVENUE- 10TH FL., NEW YORK, NY, 10151, US

(73) Assignee: Oxford Biomedica (UK) Limited, Oxford (GB)

(21) Appl. No.: 11/692,846

(22) Filed: Mar. 28, 2007

Related U.S. Application Data
(63) Continuation of application No. 10/255,031, filed on Sep. 23, 2002, now abandoned.
(60) Provisional application No. 60/330,659, filed on Oct. 26, 2001.

(30) Foreign Application Priority Data
Sep. 21, 2001 (GB) 0122803.0

(51) Int. Cl. A61K 31/711; A61K 31/7105; A61K 35/76; A61P 35/00

(52) U.S. Cl. 514/44

(57) ABSTRACT

The present invention relates to a retroviral vector system comprising a therapeutic gene wherein said retroviral vector system is pseudotyped with at least part of a heterologous envelope protein or a mutant, variant or homologue thereof and wherein said therapeutic gene is downstream of an internal promoter.

### CROSS-REFERENCE TO RELATED APPLICATIONS

This application is a continuation of U.S. patent application Ser. No. 10/255,031, filed Sep. 23, 2002, which claims benefit of priority to U.S. Provisional Patent Application 60/330,659, filed Oct. 26, 2001, listing Andrew Slade and Susan Kingman as inventors, and to U.K. Patent Application 0122803.0, filed Sep. 21, 2001, listing Oxford Biomedica (UK) Limited as applicant; each of these three applications is hereby incorporated by reference in its entirety.

### FIELD OF THE INVENTION

The present invention relates to a retroviral vector system. In particular, the present invention relates to a retroviral vector system capable of delivering a therapeutic gene to a target cell, for the treatment of cancer.

### BACKGROUND OF THE INVENTION

Gene therapy is a method of treating disease by the introduction of genes into human cells rather than by the administration of chemical agents or proteins. Initially envisaged as a treatment for monogenic inherited disease its application has broadened to include any disease that could benefit from the inhibition or addition of a functional gene to target cells. The most commonly used delivery vehicles are retroviruses but other viruses and various DNA formulations are also being used. A wide range of human and foreign genes have been introduced into patients. These include bacterial and viral markers such as Escherichia coli Neo and lacZ marker genes and herpes simplex virus thymidine kinase genes, human cytokine genes, growth factors, tumour antigen genes, tumour suppressor genes, immune co-stimulatory genes and genes to correct inherited disorders such as cystic fibrosis transmembrane conductance regulator (CFTR).

Retroviruses are RNA viruses with a life cycle different to that of lytic viruses. In this regard, a retrovirus is an infectious entity that replicates through a DNA intermediate. When a retrovirus infects a cell, its genome is converted to a DNA form by a reverse transcriptase enzyme. The DNA copy serves as a template for the production of new RNA genomes and virally encoded proteins necessary for the assembly of infectious viral particles.

One application of gene therapy is in the treatment of cancer—such as breast cancer. Breast cancer is the most common cancer type in women and the most common cause of death in women between the ages of 35 and 54 years. Breast cancer occurs in 1 in 9 American women. In the Netherlands, it accounts for 22% of all cancer deaths and, in women, it is the most common cause of cancer related mortality after lung cancer. In general, the 10 year survival rate in so-called node negative patients treated only with surgery is about 70% whereas, in node positive patients, 10 year survival drops to 30%. Overall, however, after 10 years, less than 10% of patients benefit in terms of mortality rates from adjuvant chemotherapy.

The incidence of breast cancer is rising, to some extent perhaps due to better and earlier diagnosis and elimination of other forms of lethal disease in young women. Mortality rates have remained much the same. However, improved diagnosis and increased longevity are unlikely to account for the entire increase in the incidence of breast cancer. The number of new cases diagnosed world wide in the year 2000 is predicted to be between 1.1 and 1.4 million. Some of the other factors involved include age, race, radiation exposure, and onset of menarche and menopause, obesity and unknown environmental causes. Long term use of hormone replacement therapy has also been linked to an increased incidence of breast cancer. Women in Far Eastern countries have only one-seventh the risk of developing breast cancer by comparison with the Western world. Interestingly, this advantage is lost when such populations migrate to Western civilisations, strongly supporting the hypothesis of causative environmental factors. By contrast, hereditary factors appear only to account for between 5-8% of the overall incidence of breast cancer, although they may predispose to its development at an early age. Increased risks have also been observed in first degree relatives of patients who developed pre-menopausal breast cancer.

A number of strategies have been identified for the direct destruction of tumours by gene therapy. The principal strategies are, (i) direct molecular intervention, for example the replacement of mutant or absent tumour suppressor genes such as p53 in tumour cells, (ii) immune modulation for example by providing cytokines, antigens or co-stimulatory molecules to the tumour cells or the tumour environment to induce immunological rejection of the tumour, (iii) the production of tumour specific toxins for example antibody-ricin fusions, (iv) the expression of suicide genes which are generally enzymes that can activate prodrugs to produce cytotoxins and (v) the expression of anti-angiogenic proteins such as endostatin.

The present inventors have previously developed a retroviral vector system for the delivery of a therapeutic gene to a target cell. The present invention provides another retroviral vector system with changes at the molecular level.

Thus, the present invention relates to improvements in retroviral vector systems.

### BRIEF SUMMARY OF THE INVENTION

The present invention is based upon the surprising finding that when a retroviral vector system pseudotyped with at least part of a heterologous envelope protein or a mutant, variant or homologue thereof and comprising a therapeutic gene under the control of an internal promoter is used to deliver a therapeutic gene, an increased level of expression of the therapeutic gene and an increased gene transfer efficiency is achievable even when using concentrated stocks of vector.

In a first aspect, the invention provides a retroviral vector system capable of delivering a therapeutic gene to a target cell wherein said retroviral vector system is pseudotyped with at least part of a heterologous envelope protein or a mutant, variant or homologue thereof wherein the transcription of said therapeutic gene is under the control of an internal promoter.

Preferably, the internal promoter is a cytomegalovirus promoter.

Preferably, the expression product(s) encoded by the therapeutic gene encode a pro-drug activating enzyme.

Indeed according to a second aspect, the invention provides a retroviral vector system capable of delivering a therapeutic gene to a target cell wherein said retroviral vector system is pseudotyped with at least part of a heterologous envelope protein or a mutant, variant or homologue thereof wherein the therapeutic gene is capable of encoding a pro-drug activating enzyme.

Preferably, the pro-drug activating enzyme is cytochrome P450 2B6.

Preferably, the pro-drug is cyclophosphamide or ifosfamide.

Preferably, the heterologous envelope protein is at least part of RD114 or a mutant, variant or homologue thereof.

Indeed, according to a third aspect, the invention provides a retroviral vector system capable of delivering a therapeutic gene to a cancer cell, wherein the retroviral vector system is pseudotyped with at least part of RD114 or a mutant, variant or homologue thereof.

In a fourth aspect, there is provided a retroviral vector particle obtainable from such a retroviral vector system.

In a fifth aspect, there is provided a retroviral vector genome suitable for use in preparing such a retroviral vector system.

In a sixth aspect, there is provided a producer cell capable of producing such a retroviral vector particle.

In a seventh aspect, there is provided a target cell transfected or transduced with such a retroviral vector system.

In an eighth aspect, there is provided a kit comprising either a retroviral vector genome and one or more producer plasmids and optionally a cell to be transfected or the retroviral vector genome and one or more packaging cells.

In a ninth aspect, there is provided a method of transducing a target cell with one or more therapeutic genes comprising the step of transducing said target cell with a retroviral vector system according to the present invention.

In a tenth aspect, there is provided a method for treating or preventing a disease in a subject, which comprises the step of administering such a retroviral vector system. Preferably, the retroviral vector system is administered by the intratumoral route.

In an eleventh aspect, there is provided a pharmaceutical composition comprising a therapeutically effective amount of such a retroviral vector system, a retroviral particle, a retroviral vector genome, and/or a producer cell and a pharmaceutically acceptable carrier, diluent, excipient or adjuvant or any combination thereof.

In a twelfth aspect, there is provided such a retroviral vector system, a retroviral particle, a retroviral vector genome and/or a producer cell for use in medicine.

In an thirteenth aspect, there is provided the use of such a retroviral vector system, a retroviral particle, a retroviral vector genome and/or a producer cell in the manufacture of a pharmaceutical composition for the treatment of a disease.

### BRIEF DESCRIPTION OF THE SEVERAL VIEWS OF THE DRAWING(S)

FIG. 1A is a diagrammatic representation of the vector genomes of OB80 and OB83. In OB80, P450 is expressed from the 5′ LTR; translation of lacZ is ires-dependent; G418 selection for the NeoR function maintains its expression level from the internal SV40 promoter, but does not select for the full length genome under the control of the 5′ LTR. In OB83, P450 is expressed from an internal CMV promoter and the expression level is 4-fold higher than in OB80; translation of lacZ is no longer ires-dependent. This improves the ability to track the vector in tissue sections; NeoR is expressed as a lacZ fusion protein. Selection for this function using G418 impacts directly on the expression level of the full length genome. NB: In order to produce the vector particles the genome plasmids are introduced into the FLY packaging cell lines via an intermediate passage through HEK293 cells. The genome configuration of the resulting retroviral vector that is released from the producer cell line is predictably rearranged without the acquisition of any new sequences according to the mechanism of reverse transcription. In this way the MoMLV LTR replaces the CMV-LTR at the 5′ end of the vector genome as it appears in the producer (and target cells). It is this configuration that is shown in the figure.

FIG. 1B is a map of the OB80 genome plasmid with a key to the derivation of its sequence.

FIG. 2 shows the synthetic linker used to make the β-Geo fusion. The EcoR1 and Xma111 sticky ends are shown underlined as are the last residue of lacZ and the first residue of NeoR. A flexible linker that joins the two functional moieties is shown in bold type.

FIG. 3 is a diagrammatic representation of the pOB83 cloning scheme.

FIG. 4 is a plasmid map of the pOB83 genome illustrating the key features.

FIG. 5 illustrates the derivation of the FLY retroviral packaging cell lines.

FIG. 6 shows the results of the PCR analysis to detect the integrated VSV-G coding sequence in the RD/83 retroviral producer cell line.

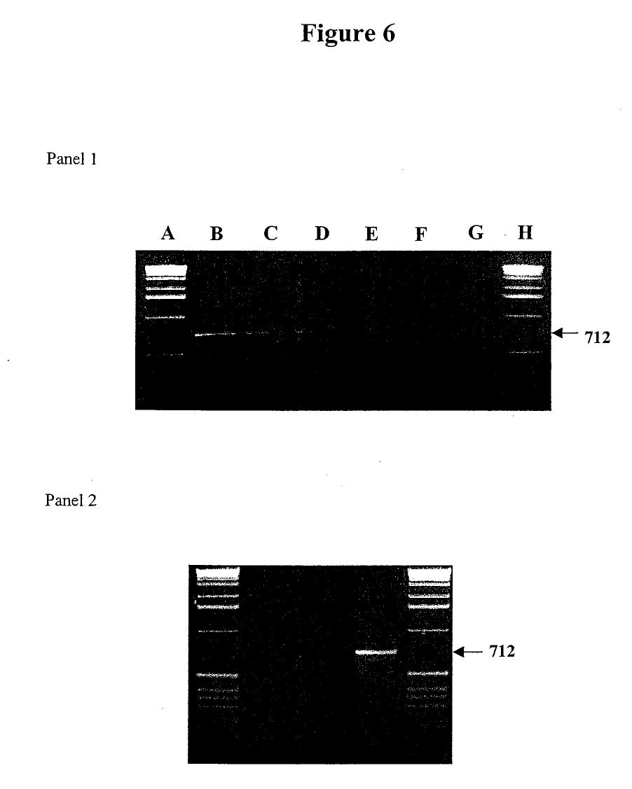

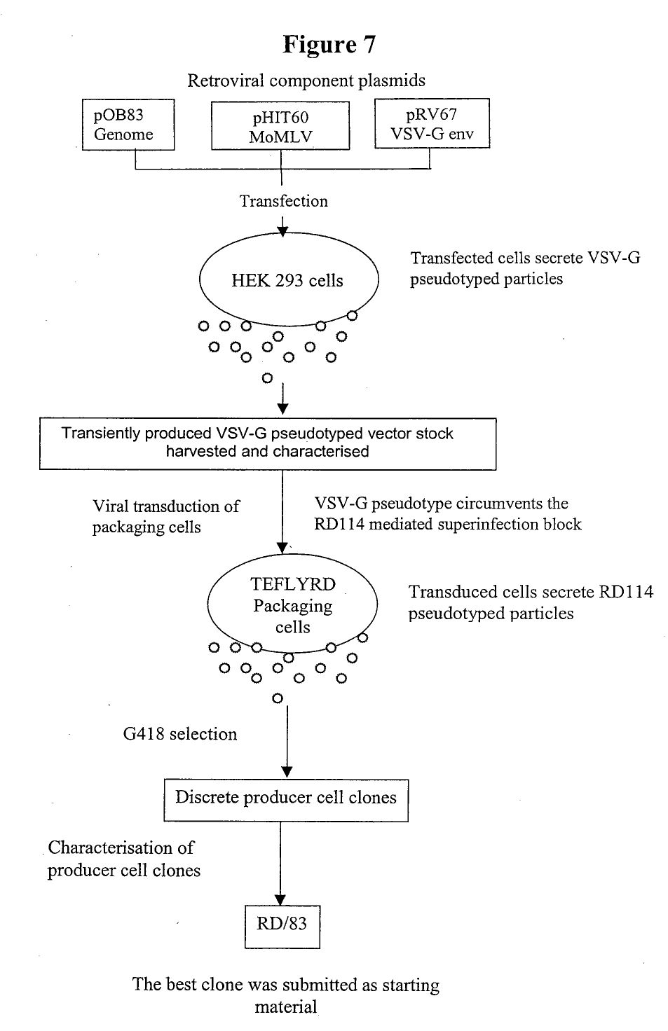

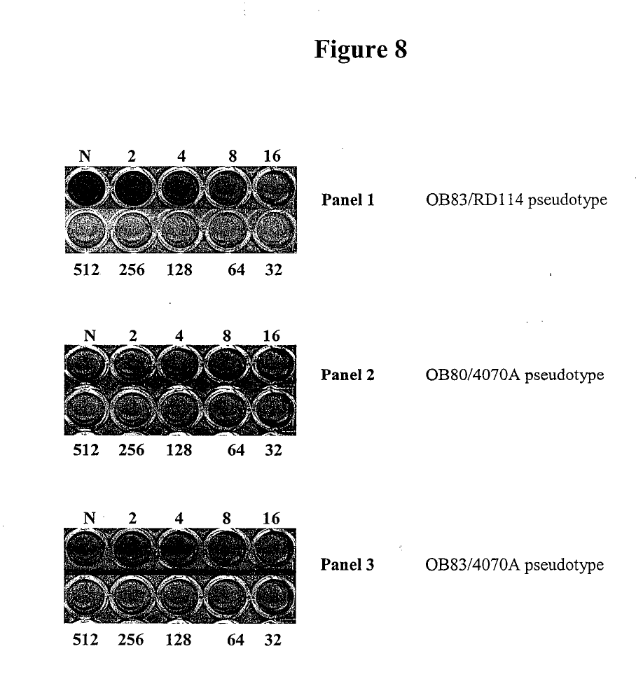

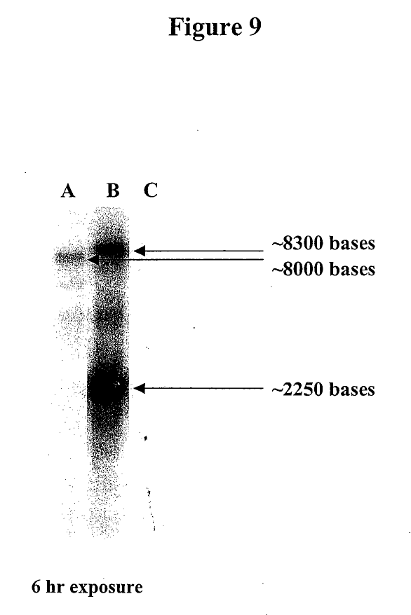

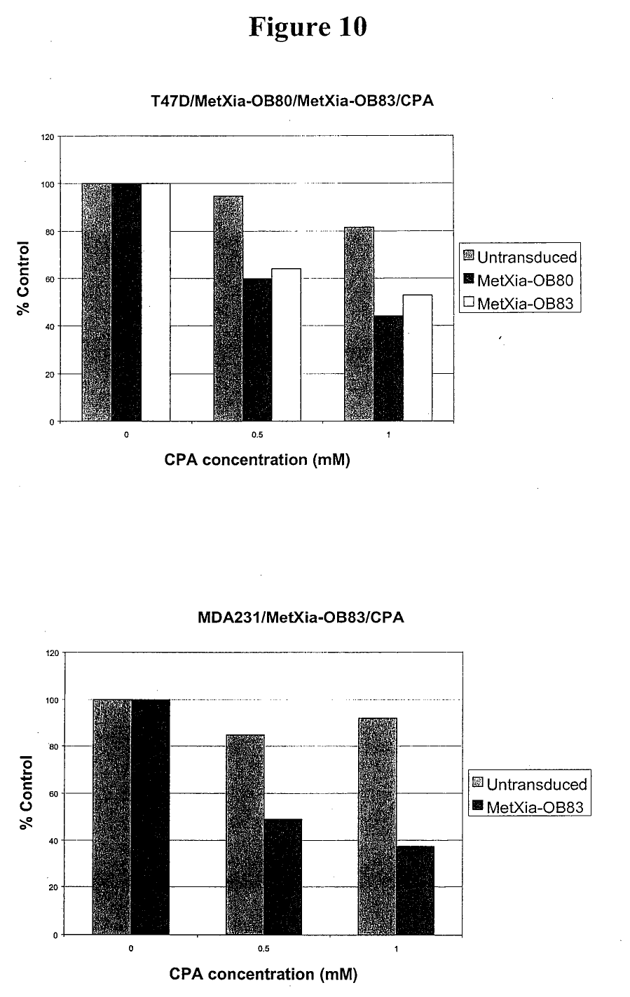

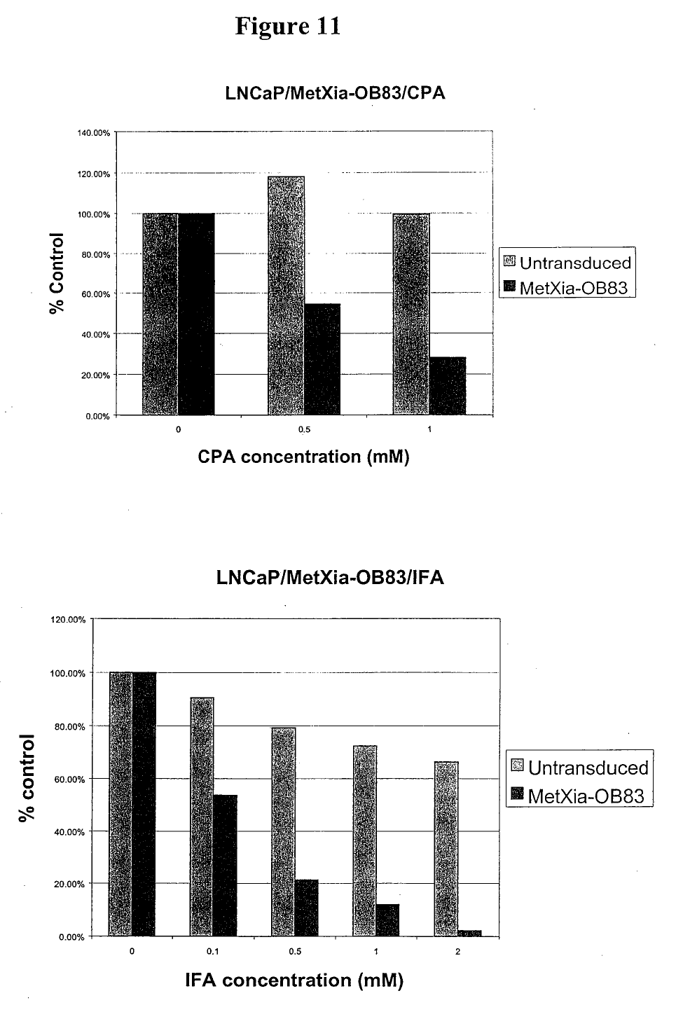

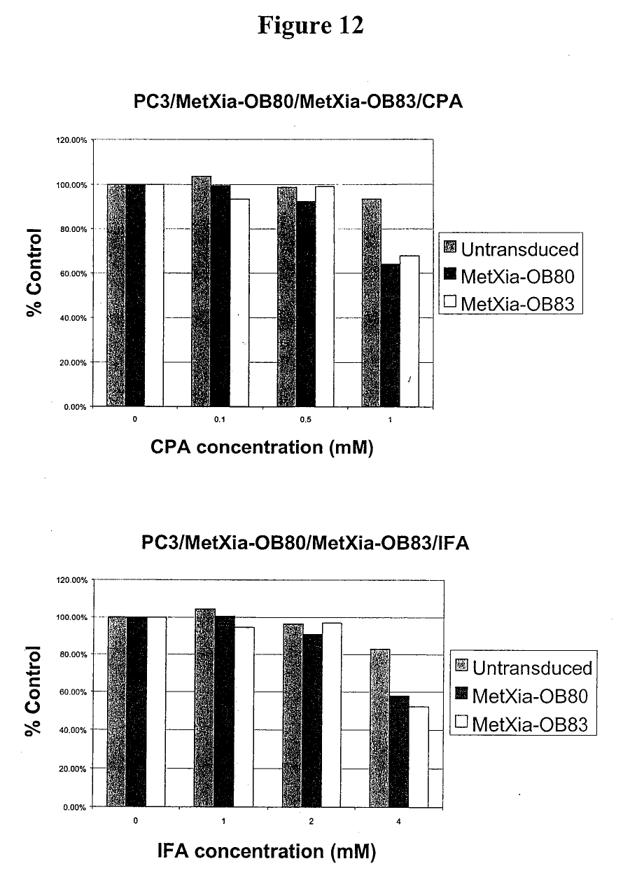

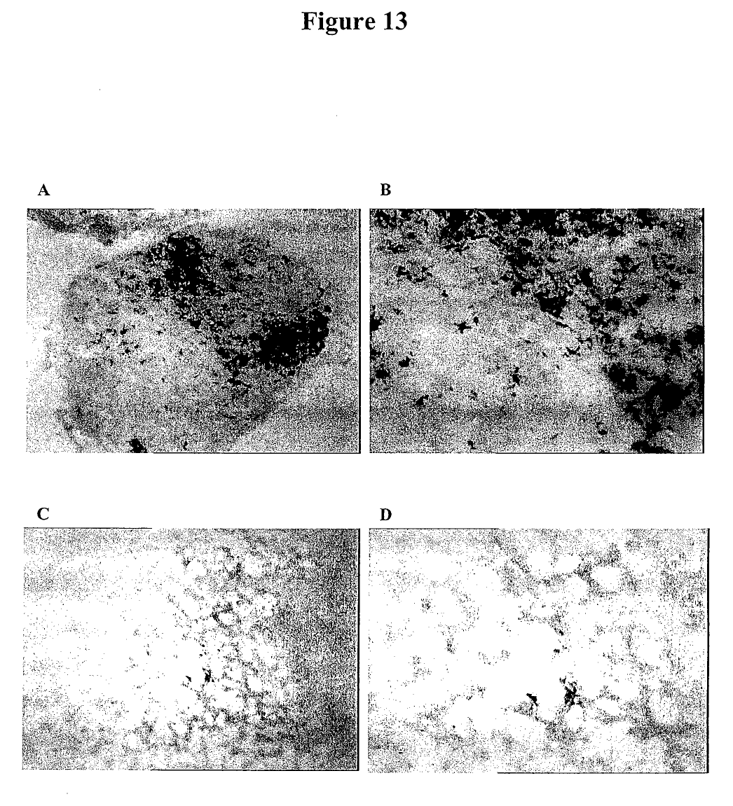

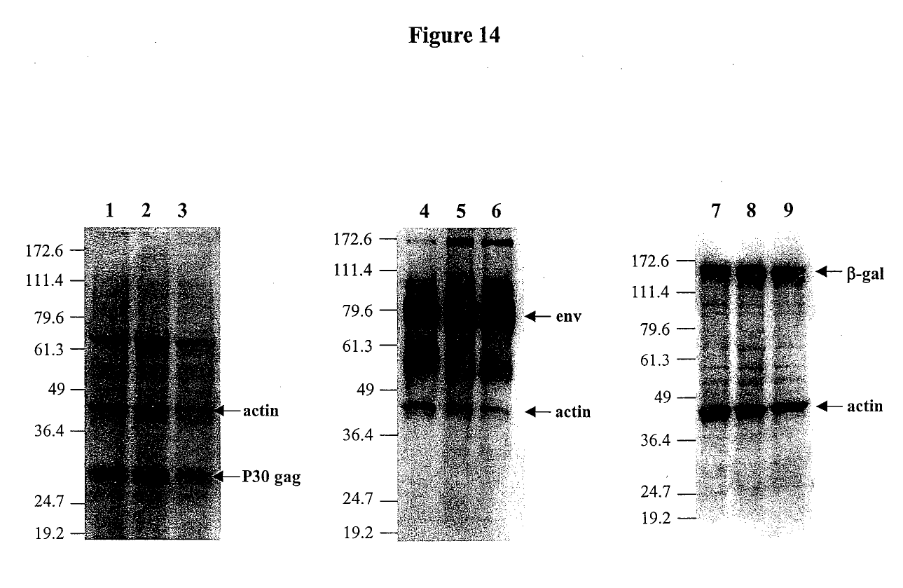

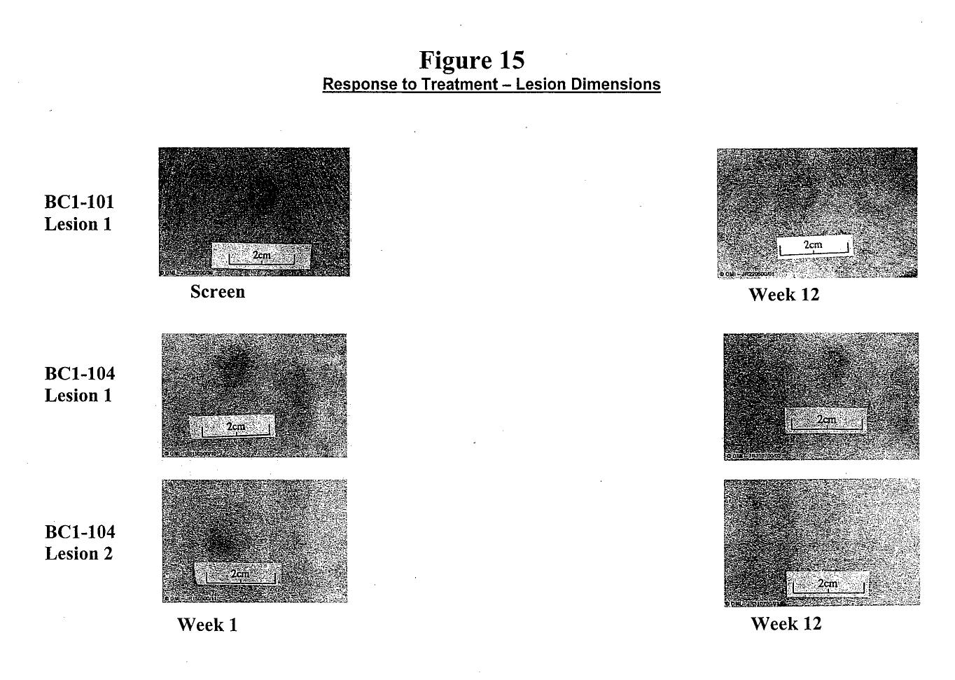

Panel 1 of FIG. 6 shows the sensitivity of the PCR assay. The VSV-G encoding plasmid pRV67 was serially diluted in 10 ng of RD/83 genomic DNA and then subjected to PCR amplification. The starting copy numbers were as follows. Lane B=130,000, Lane C=13,000, Lane D=1,300, Lane E=130, Lane F=13 and Lane G=1.3 copies. Lanes A and H are 1 kb marker ladder. It can be seen that a band of the expected 712 bp is clearly visible down to 130 copies and is present at a reduced level where the starting copy number was 13 molecules. The sensitivity of the assay is therefore set at between 1e3 and 130 copies.

Panel 2 of FIG. 6 shows the results of the amplification of RD/83 genomic DNA. Lane B is 10 ng of RD/83 genomic DNA. Lane C is the no DNA control and Lane D is a positive control consisting of 1000 copies of pRV67 in 100 ng of RD/83 DNA. Lanes A and E are 1 kb marker ladder. No signal was detected in the RD/83 genomic DNA reaction. The results from the control tubes show that the assay was valid.

FIG. 7 is a process flow chart illustrating the derivation of the RD/83 producer cell line.

FIG. 8 shows the transducing power of three two-fold dilution series of different concentrated retroviral vector stocks that have been used to transduce HT1080 target cells and then visualised by X-Gal staining. The fold dilution is shown in bold type, above or below each individual well.

Panel 1 shows the efficient transduction of HT1080 cells by concentrated OB83/RD114 (OB83 pseudotyped with RD114). The number of cells transduced in the neat and two-fold diluted wells is >50% and is clearly visible to the naked eye, characterised by strong non-IRES mediated expression of lacZ (B-geo). The level of transduction decreases with increasing dilution.

In contrast the transduction of HT1080 cells by OB80/4070A (OB80 pseudotyped with 4070A), as shown in panel 2, is not visible. Microscopic examination of the wells showed that the peak level of transduction occurred when the material was diluted four-fold. At this point the number of cells transduced was ˜20% and they showed characteristically weak IRES mediated expression of lacZ.

Panel 3 shows the results obtained with the “hybrid vector” which has the OB83 genome and the 4070A pseudotype. The higher lacZ expression level obtained from this genome allows the transduced cells to be seen in this image. As with OB80/4070A, the peak level of transduction (˜20%) was seen at a four-fold dilution and after this, transduction levels decreased with increased dilution.

FIG. 9 represents the Northern blot analysis of P450 expression levels from OB80 and OB83. Lane A=OB80 transduced tumor cells showing a single P450 specific transcript of ˜8000 bases. Lane B=OB83 transduced tumour cells showing two P450 containing transcripts of ˜8300 and ˜2250 bases. The shorter transcript which is directed by the internal CMV promoter is present in four-fold excess over the LTR directed transcript. Lane C=Mock transduced tumour cells showing no P450 specific transcripts.

FIG. 10 represents the in vitro potency of OB83/RD114, OB80/4070A and untransduced cells in breast cancer cell lines.

FIG. 11 represents the in vitro potency of OB83/RD114 and untransduced cells in the LNCaP prostate cancer cell line.

FIG. 12 represents the in vitro potency of OB83/RD114, OB80/4070A and untransduced cells in the PC3 prostate cancer cell line.

FIG. 13 represents the in vivo gene transfer in MDA231 tumour xenografts showing tissue sections from OB80/4070A and OB83/RD114 injected MDA231 tumour xenografts that have been stained with X-Gal to visualise any transduced cells. Panel A shows a representative section from a OB83/RD114 treated tumour photographed at 4× magnification; Panel B shows the same section as in panel A, photographed at 10× magnification; Panel C shows a representative section from a OB80/4070A treated tumour photographed at 10× magnification; Panel D shows the same section as in panel C, photographed at 20× magnification.

FIG. 14 illustrates the Western blot analysis of producer cell component expression. Lanes 1, 2 and 3 show samples of cell lysate at time zero, 3 months+g418 and 3 months−g418 respectively that have been probed with an anti-gag P30 antibody. The expression level in all three samples is equal; Lanes 4, 5 and 6 show samples of cell lysate at time zero, 3 months+g418 and 3 months−g418 respectively that have been probed with an anti-RD144 gp70 antibody. The expression level in all three samples is equal; Lanes 7, 8 and 9 show samples of cell lysate at time zero, 3 months+g418 and 3 months−g418 respectively that have been probed with an anti-βgal antibody. The expression level in all three samples is equal; the actin controls show an equal loading of protein in all cases.

FIG. 15 shows photographs of patient lesions at time points 1 week and 12 weeks after treatment for patient BC1-104 and before treatment (week 0) and 12 weeks after treatment for patient BC1-101 in a phase I/II clinical trial involving OB80/4070A.

### DETAILED DESCRIPTION OF THE INVENTION

Retroviruses

The concept of using viral vectors for gene therapy is well known (Verma and Somia (1997) Nature 389:239-242).

There are many retroviruses including murine leukemia virus (MLV), human immunodeficiency virus (HIV), equine infectious anaemia virus (EIAV), mouse mammary tumour virus (MMTV), Rous sarcoma virus (RSV), Fujinami sarcoma virus (FuSV), Moloney murine leukemia virus (Mo-MLV), FBR murine osteosarcoma virus (FBR MSV), Moloney murine sarcoma virus (Mo-MSV), Abelson murine leukemia virus (A-MLV), Avian myelocytomatosis virus-29 (MC29), and Avian erythroblastosis virus (AEV). In a preferred embodiment the vector system of the present invention is derivable from the Mo-MLV.

A detailed list of retroviruses may be found in Coffin et al (“Retroviruses” 1997 Cold Spring Harbour Laboratory Press Eds: J M Coffin, S M Hughes, H E Varmus pp 758-763).

During the process of infection, a retrovirus initially attaches to a specific cell surface receptor. On entry into the susceptible host cell, the retroviral RNA genome is then copied to DNA by the virally encoded reverse transcriptase which is carried inside the parent virus. This DNA is transported to the host cell nucleus where it subsequently integrates into the host genome. At this stage, it is typically referred to as the provirus. The provirus is stable in the host chromosome during cell division and is transcribed like other cellular genes. The provirus encodes the proteins and other factors required to make more virus, which can leave the cell by a process sometimes called “budding”.

Each retroviral genome comprises genes called gag, pol and env, which code for viral proteins and enzymes. These genes are flanked at both ends by regions called long terminal repeats (LTRs). The LTRs are responsible for proviral integration, and transcription. They also serve as enhancer-promoter sequences. In other words, the LTRs can control the expression of the viral genes. Encapsidation of the retroviral RNAs occurs by virtue of a psi sequence located at the 5′ end of the viral genome.

The LTRs themselves are identical sequences that can be divided into three elements, which are called U3, R and U5. U3 is derived from the sequence unique to the 3′ end of the RNA. R is derived from a sequence repeated at both ends of the RNA and U5 is derived from the sequence unique to the 5′ end of the RNA. The sizes of the three elements can vary considerably among different retroviruses.

For the viral genome, the site of transcription initiation is at the boundary between U3 and R in one LTR and the site of poly (A) addition (termination) is at the boundary between R and U5 in the other LTR. U3 contains most of the transcriptional control elements of the provirus, which include the promoter and multiple enhancer sequences responsive to cellular and in some cases, viral transcriptional activator proteins. Some retroviruses have any one or more of the following genes that code for proteins that are involved in the regulation of gene expression: tat, rev, tax and rex.

With regard to the structural genes gag, pol and env, gag encodes the internal structural protein of the virus. Gag protein is proteolytically processed into the mature proteins MA (matrix), CA (capsid) and NC (nucleocapsid). The pol gene encodes the reverse transcriptase (RT), which contains DNA polymerase, associated RNaseH and integrase (IN), which mediate replication of the genome. The env gene encodes the surface (SU) glycoprotein and the transmembrane (TM) protein of the virion, which form a complex that interacts specifically with cellular receptor proteins. This interaction leads ultimately to infection by fusion of the viral membrane with the cell membrane.

Vector Systems

Retroviral vector systems have been proposed as a delivery system for inter alia the transfer of a nucleotide sequence of interest to one or more sites of interest. The transfer can occur in vitro, ex vivo, in vivo, or in combinations thereof. Retroviral vector systems have even been exploited to study various aspects of the retrovirus life cycle, including receptor usage, reverse transcription and RNA packaging (reviewed by Miller, 1992 Curr Top Microbiol Immunol 158:1-24).

As used herein the term “vector system” means a vector particle capable of transducing a recipient cell with a therapeutic gene.

A vector particle includes the following components: a vector genome, which may contain one or more therapeutic genes, a nucleocapsid encapsidating the nucleic acid, and a membrane surrounding the nucleocapsid.

The term “nucleocapsid” refers to at least the group specific viral core proteins (gag) and the viral polymerase (pol) of a retrovirus genome. These proteins encapsidate the packagable sequences and are further surrounded by a membrane containing an envelope glycoprotein.

Once within the cell, the RNA genome from a retroviral vector particle is reverse transcribed into DNA and integrated into the DNA of the recipient cell.

As used herein, the term “vector genome” refers to both to the RNA construct present in the retroviral vector particle and the integrated DNA construct. The term also embraces a separate or isolated DNA construct capable of encoding such an RNA genome. A retroviral genome comprises at least one component part derivable from a retrovirus—such as murine leukemia virus (MLV), human immunodeficiency virus (HIV), equine infectious anaemia virus (EIAV), mouse mammary tumour virus (MMTV), Rous sarcoma virus (RSV), Fujinami sarcoma virus (FuSV), Moloney murine leukemia virus (Mo-MLV), FBR murine osteosarcoma virus (FBR MSV), Moloney murine sarcoma virus (Mo-MSV), Abelson murine leukemia virus (A-MLV), Avian myelocytomatosis virus-29 (MC29), and Avian erythroblastosis virus (AEV).

Preferably, the retroviral genome comprises at least one component part derivable from a Moloney murine leukaemia virus (MoMLV) (GenBank accession nos. J02255 J02256 J02257 M76668).

The term “derivable” is used in its normal sense as meaning a nucleotide sequence or a part thereof which need not necessarily be obtained from a virus but instead could be derived therefrom. By way of example, the sequence may be prepared synthetically or by use of recombinant DNA techniques.

The viral vector genome is preferably “replication defective” by which we mean that the genome does not comprise sufficient genetic information alone to enable independent replication to produce infectious viral particles within the recipient cell. Preferably, the viral genome lacks a functional env, gag or pol gene. More preferably, the genome lacks env, gag and pol genes.

The viral vector genome comprises some or all of the long terminal repeats (LTRs). Preferably the genome comprises at least part of the LTRs or an analogous sequence which is capable of mediating proviral integration, and transcription. More preferably, the genome comprises a Cytomegalovirus LTR and a MoMLV LTR. Most preferably, the genome comprises a Cytomegalovirus 5′ LTR and a MoMLV 3′ LTR.

The LTRs may also comprise or act as enhancer-promoter sequences.

The viral vector system of the present invention also comprises a therapeutic gene under the control of an internal promoter.

The term “internal promoter” is used herein to indicate a promoter which is distinct from the viral promoter sequences found in the LTRs. Preferably the internal promoter is immediately upstream of the therapeutic gene.

Suitable promoting sequences are preferably strong promoters derived from the genomes of viruses—such as polyoma virus, adenovirus, fowlpox virus, bovine papilloma virus, avian sarcoma virus, cytomegalovirus (CMV), a retrovirus and Simian Virus 40 (SV40)—or from heterologous mammalian promoters—such as the actin promoter or ribosomal protein promoter. Transcription of a gene may be increased further by inserting an enhancer sequence into the vector. Enhancers are relatively orientation and position independent. However, one will typically employ an enhancer from a eukaryotic cell virus—such as the SV40 enhancer on the late side of the replication origin (bp 100-270) and the CMV early promoter enhancer. The enhancer may be spliced into the vector at a position 5′ or 3′ to the promoter, but is preferably located at a site 5′ from the promoter.

In a preferred embodiment, the internal promoter is a cytomegalovirus (CMV) promoter. By using an internal CMV promoter, the present inventors have found that the level of expression of the therapeutic gene can be increased as much as 4-fold when compared to the expression from a 5′ LTR. Thus, advantageously the potency of the therapeutic gene product may be increased.

In a preferred embodiment, the retroviral vector system also comprises a packaging signal to enable the genome to be packaged into a vector particle in a producer cell. The term “packaging signal” is used in reference to the non-coding, cis-acting sequence required for encapsidation of retroviral RNA strands during viral particle formation or an analogous component which is capable of causing encapsidation. In HIV-1, this sequence has been mapped to loci extending from upstream of the major splice donor site (SD) to at least the gag start codon. Preferably, the packaging signal is psi or an analogous component, which is capable of causing encapsidation.

Selection/Marker Genes

Preferably, the retroviral vector genome further comprises a selectable marker in order to select cells that are producing vectors at high titre. Many different selectable markers have been used successfully in retroviral vectors. These are reviewed in “Retroviruses” (1997 Cold Spring Harbour Laboratory Press Eds: J M Coffin, S M Hughes, H E Varmus pp 444) and include, but are not limited to, the bacterial neomycin and hygromycin phosphotransferase genes which confer resistance to G418 and hygromycin respectively; a mutant mouse dihydrofolate reductase gene which confers resistance to methotrexate; the bacterial gpt gene which allows cells to grow in medium containing mycophenolic acid, xanthine and aminopterin; the bacterial hisD gene which allows cells to grow in medium without histidine but containing histidinol; the multidrug resistance gene (mdr) which confers resistance to a variety of drugs; and the bacterial genes which confer resistance to puromycin or phleomycin. Preferably, the selectable marker is a gene capable of encoding resistance to G418 (NeoR) which has previously been introduced into patients with no adverse effects.

Preferably, the retroviral vector system of the present invention also comprises an identifiable marker to assess gene transfer efficiency and gene expression. Preferably, the identifiable marker is a gene capable of encoding β-galactosidase (lacZ). This gene has previously been introduced into patients with no adverse effects. Transduced cells and tissues may be visualised using immunohistochemical staining—such as staining with X-gal. Advantageously, the therapeutic gene—such as cytochrome p450—may also be used as an identifiable marker to identify transduced cells and tissues.

Transduced cells and tissues may be visualised as follows. Briefly, frozen tumour samples are sectioned using a cryostat and mounted on slides. Sections are then probed for the presence of products of the therapeutic gene (P450 2B6) and for the marker gene (lacZ). Expression of the marker gene is determined using two approaches. In the first instance, the assessment for β-galactosidase expression in the frozen section is by X-gal histochemistry. This assay uses the β-galactosidase cleavage of a substrate, X-gal, to produce an insoluble indigo precipitate in the presence of ferrous ions. If confirmatory data are required, the lacZ gene product can also be detected by immunohistochemical methods. Sections are fixed, desiccated and incubated with a primary anti-β-galactosidase antibody. Unbound primary antibody is rinsed off prior to addition of secondary detection reagents.

The immunohistochemical detection of cytochrome P450 2B6 is performed in an analogous way to that for β-galactosidase as described above using the appropriate primary and secondary detecting antibodies.

Preferably, the selectable marker and the identifiable marker are expressed as a fusion protein. More preferably, expression of the fusion protein is under the control of a 5′ CMV LTR that also regulates the expression of psi such that selection of the vector using G418 impacts directly on the expression level of the full length genome.

The retroviral vector genome of the present invention may also comprise suitable insertion sites—such as restriction enzyme sites—for inserting one or more therapeutic genes.

Pseudotyping

In the design of retroviral vector systems it is desirable to engineer particles with different target cell specificities to the native virus, to enable the delivery of genetic material to an expanded or altered range of cell types. One manner in which to achieve this is by engineering the virus envelope protein to alter its specificity. Another approach is to introduce a heterologous envelope protein into the vector particle to replace or add to the native envelope protein of the virus.

The term “pseudotyping” means incorporating in at least a part of, or substituting a part of, or replacing all of, an env gene of a viral genome with a heterologous env gene, for example an env gene from another virus. Pseudotyping is not a new phenomenon and examples may be found in WO 99/61639, WO-A-98/05759, WO-A-98/05754, WO-A-97/17457, WO-A-96/09400, WO-A-91/00047 and Mebatsion et al 1997 Cell 90, 841-847.

Pseudotyping can improve retroviral vector stability and transduction efficiency. A pseudotype of murine leukemia virus packaged with lymphocytic choriomeningitis virus (LCMV) has been described (Miletic et al (1999) J. Virol. 73:6114-6116) and shown to be stable during ultracentrifugation and capable of infecting several cell lines from different species.

The vector system described herein is pseudotyped with at least part of a heterologous envelope protein or a mutant, variant or homologue thereof. Suitable heterologous envelope proteins may include at least part of the MLV envelope protein or a mutant, variant or homologue thereof which is capable of pseudotyping a variety of different retroviruses. MLV envelope proteins from an amphotropic virus allow transduction of a broad range of cell types including human cells. Another suitable envelope protein may include at least part of the envelope glycoprotein (G) of Vesicular stomatitis virus (VSV) or a mutant, variant or homologue thereof. VSV is a rhabdovirus, which has an envelope protein that has been shown to be capable of pseudotyping certain retroviruses. Its ability to pseudotype MoMLV-based retroviral vectors in the absence of any retroviral envelope proteins was first shown by Emi et al (1991) Journal of Virology 65:1202-1207. Another suitable envelope protein may include at least part of the envelope of gibbon ape leukaemia virus (GaLV) or a mutant, variant or homologue thereof.

Preferably, the heterologous envelope protein is at least part of RD114 or a mutant, variant or homologue thereof from the RD114/simian type D retroviruses. RD114 is discussed in more detail below.

RD114

The RD114/simian type D retroviruses include the feline endogenous retrovirus RD114, all strains of simian immunosupressive type D retroviruses, the ovian reticuloendotheliosis group including spleen necrosis virus and the baboon endogenous virus. All of these viruses use a common cell surface receptor for cell entry called RD114. The receptor for members of the RD114/type D retrovirus interference group in humans has been identified and cloned (Rasko et al. (1999) Proc. Natl. Acad. Sci. 96 2129-2134). A single ORF encoding the receptor is localised within human 19q13.3. The receptor functions as a neutral amino acid transporter and infection of human cells with replication-competent viruses of the RD114/type D retrovirus interference group reduces uptake of neutral amino acids.

In one aspect, the present invention surprisingly demonstrates that when a retroviral vector system is pseudotyped with the envelope protein of RD114 it is possible to get high levels of gene transfer even when using concentrated stocks of vector. Preferably, 50% or more target cells are transduced when concentrated vector stocks are used.

The sequence of the RD114 env gene is X87829 and is publicly available on the EMBL database.

Mutants, Variants and Homologues

The retroviral vector system used in the present invention may be pseudotyped with at least part of a heterologous envelope protein or a mutant, variant or homologue thereof.

The term “wild type” is used to mean a polypeptide having a primary amino acid sequence which is identical with the native protein (i.e., the viral protein).

The term “mutant” is used to mean a polypeptide having a primary amino acid sequence which differs from the wild type sequence by one or more amino acid additions, substitutions or deletions. A mutant may arise naturally, or may be created artificially (for example by site-directed mutagenesis). Preferably the mutant has at least 90% sequence identity with the wild type sequence. Preferably the mutant has 20 mutations or less over the whole wild-type sequence. More preferably the mutant has 10 mutations or less, most preferably 5 mutations or less over the whole wild-type sequence.

The term “variant” is used to mean a naturally occurring polypeptide which differs from a wild-type sequence. A variant may be found within the same viral strain (i.e. if there is more than one isoform of the protein) or may be found within a different strain. Preferably the variant has at least 90% sequence identity with the wild type sequence. Preferably the variant has 20 mutations or less over the whole wild-type sequence. More preferably the variant has 10 mutations or less, most preferably 5 mutations or less over the whole wild-type sequence.

Here, the term “homologue” means an entity having a certain homology with the wild type amino acid sequence and the wild type nucleotide sequence. Here, the term “homology” can be equated with “identity”.

In the present context, an homologous sequence is taken to include an amino acid sequence which may be at least 75, 85 or 90% identical, preferably at least 95 or 98% identical to the subject sequence. Typically, the homologues will comprise the same active sites etc. as the subject amino acid sequence. Although homology can also be considered in terms of similarity (i.e. amino acid residues having similar chemical properties/functions), in the context of the present invention it is preferred to express homology in terms of sequence identity.

In the present context, an homologous sequence is taken to include a nucleotide sequence which may be at least 75, 85 or 90% identical, preferably at least 95 or 98% identical to the subject sequence. Typically, the homologues will comprise the same sequences that code for the active sites etc. as the subject sequence. Although homology can also be considered in terms of similarity (i.e. amino acid residues having similar chemical properties/functions), in the context of the present invention it is preferred to express homology in terms of sequence identity.

Homology comparisons can be conducted by eye, or more usually, with the aid of readily available sequence comparison programs. These commercially available computer programs can calculate % homology between two or more sequences.

% homology may be calculated over contiguous sequences, i.e. one sequence is aligned with the other sequence and each amino acid in one sequence is directly compared with the corresponding amino acid in the other sequence, one residue at a time. This is called an “ungapped” alignment. Typically, such ungapped alignments are performed only over a relatively short number of residues.

Although this is a very simple and consistent method, it fails to take into consideration that, for example, in an otherwise identical pair of sequences, one insertion or deletion will cause the following amino acid residues to be put out of alignment, thus potentially resulting in a large reduction in % homology when a global alignment is performed. Consequently, most sequence comparison methods are designed to produce optimal alignments that take into consideration possible insertions and deletions without penalising unduly the overall homology score. This is achieved by inserting “gaps” in the sequence alignment to try to maximise local homology.

However, these more complex methods assign “gap penalties” to each gap that occurs in the alignment so that, for the same number of identical amino acids, a sequence alignment with as few gaps as possible—reflecting higher relatedness between the two compared sequences—will achieve a higher score than one with many gaps. “Affine gap costs” are typically used that charge a relatively high cost for the existence of a gap and a smaller penalty for each subsequent residue in the gap. This is the most commonly used gap scoring system. High gap penalties will of course produce optimised alignments with fewer gaps. Most alignment programs allow the gap penalties to be modified. However, it is preferred to use the default values when using such software for sequence comparisons. For example when using the GCG Wisconsin Bestfit package the default gap penalty for amino acid sequences is −12 for a gap and −4 for each extension.

Calculation of maximum % homology therefore firstly requires the production of an optimal alignment, taking into consideration gap penalties. A suitable computer program for carrying out such an alignment is the GCG Wisconsin Bestfit package (University of Wisconsin, U.S.A.; Devereux et al., 1984, Nucleic Acids Research 12:387). Examples of other software than can perform sequence comparisons include, but are not limited to, the BLAST package (see Ausubel et al., 1999 ibid—Chapter 18), FASTA (Atschul et al., 1990, J. Mol. Biol., 403-410) and the GENEWORKS suite of comparison tools. Both BLAST and FASTA are available for offline and online searching (see Ausubel et al., 1999 ibid, pages 7-58 to 7-60). However, for some applications, it is preferred to use the GCG Bestfit program. A new tool, called BLAST 2 Sequences is also available for comparing protein and nucleotide sequences (see FEMS Microbiol Lett 1999 174(2): 247-50; FEMS Microbiol Lett 1999 177(1); 187-8 and tatiana@ncbi.nlm.nih.gov).

Although the final % homology can be measured in terms of identity, the alignment process itself is typically not based on an all-or-nothing pair comparison. Instead, a scaled similarity score matrix is generally used that assigns scores to each pairwise comparison based on chemical similarity or evolutionary distance. An example of such a matrix commonly used is the BLOSUM62 matrix—the default matrix for the BLAST suite of programs. GCG Wisconsin programs generally use either the public default values or a custom symbol comparison table if supplied (see user manual for further details). For some applications, it is preferred to use the public default values for the GCG package, or in the case of other software, the default matrix, such as BLOSUM62.

Once the software has produced an optimal alignment, it is possible to calculate % homology, preferably % sequence identity. The software typically does this as part of the sequence comparison and generates a numerical result.

The sequences may also have deletions, insertions or substitutions of amino acid residues which produce a silent change and result in a functionally equivalent substance. Deliberate amino acid substitutions may be made on the basis of similarity in polarity, charge, solubility, hydrophobicity, hydrophilicity, and/or the amphipathic nature of the residues as long as the secondary binding activity of the substance is retained. For example, negatively charged amino acids include aspartic acid and glutamic acid; positively charged amino acids include lysine and arginine; and amino acids with uncharged polar head groups having similar hydrophilicity values include leucine, isoleucine, valine, glycine, alanine, asparagine, glutamine, serine, threonine, phenylalanine, and tyrosine.

Conservative substitutions may be made, for example according to the Table below. Amino acids in the same block in the second column and preferably in the same line in the third column may be substituted for each other:

| | | |
|---|---|---|
| ALIPHATIC | Non-polar | G A P |
| | | I L V |
| | Polar - uncharged | C S T M |
| | | N Q |
| | Polar - charged | D E |
| | | K R |
| AROMATIC | | H F W Y |

The present invention also encompasses homologous substitution (substitution and replacement are both used herein to mean the interchange of an existing amino acid residue, with an alternative residue) may occur i.e. like-for-like substitution such as basic for basic, acidic for acidic, polar for polar etc. Non-homologous substitution may also occur i.e. from one class of residue to another or alternatively involving the inclusion of unnatural amino acids such as ornithine (hereinafter referred to as Z), diaminobutyric acid ornithine (hereinafter referred to as B), norleucine ornithine (hereinafter referred to as O), pyridylalanine, thienylalanine, naphthylalanine and phenylglycine.

Replacements may also be made by unnatural amino acids include; alpha* and alpha-disubstituted* amino acids, N-alkyl amino acids*, lactic acid*, halide derivatives of natural amino acids such as trifluorotyrosine*, p-Cl-phenylalanine*, p-Br-phenylalanine*, p-I-phenylalanine*, L-allyl-glycine*, β-alanine*, L-α-amino butyric acid*, L-γ-amino butyric acid*, L-α-amino isobutyric acid*, L-ε-amino caproic acid#, 7-amino heptanoic acid*, L-methionine sulfone#*, L-norleucine*, L-norvaline*, p-nitro-L-phenylalanine*, L-hydroxyproline#, L-thioproline*, methyl derivatives of phenylalanine (Phe) such as 4-methyl-Phe*, pentamethyl-Phe*, L-Phe (4-amino)#, L-Tyr (methyl)*, L-Phe (4-isopropyl)*, L-Tic (1,2,3,4-tetrahydroisoquinoline-3-carboxyl acid)*, L-diaminoproprionic acid # and L-Phe (4-benzyl)*. The notation * has been utilised for the purpose of the discussion above (relating to homologous or non-homologous substitution), to indicate the hydrophobic nature of the derivative whereas # has been utilised to indicate the hydrophilic nature of the derivative, #* indicates amphipathic characteristics.

Variant amino acid sequences may include suitable spacer groups that may be inserted between any two amino acid residues of the sequence including alkyl groups such as methyl, ethyl or propyl groups in addition to amino acid spacers such as glycine or β-alanine residues. A further form of variation, involves the presence of one or more amino acid residues in peptoid form, will be well understood by those skilled in the art. For the avoidance of doubt, “the peptoid form” is used to refer to variant amino acid residues wherein the α-carbon substituent group is on the residue's nitrogen atom rather than the α-carbon. Processes for preparing peptides in the peptoid form are known in the art, for example Simon R J et al., PNAS (1992) 89(20), 9367-9371 and Horwell D C, Trends Biotechnol. (1995) 13(4), 132-134.

The term “fragment” indicates that the polypeptide comprises a fraction of the wild-type amino acid sequence. It may comprise one or more large contiguous sections of sequence or a plurality of small sections. The polypeptide may also comprise other elements of sequence, for example, it may be a fusion protein with another protein. Preferably the polypeptide comprises at least 50%, more preferably at least 65%, most preferably at least 80% of the wild-type sequence.

With respect to function, the mutant, variant or homologue should be capable of transducing target cells when used to pseudotype an appropriate vector.

Vector Titre

The practical uses of retroviral vectors have been limited largely by the titres of transducing particles which can be attained in in vitro culture (typically not more than 10⁸ particles/ml) and the sensitivity of many enveloped viruses to traditional biochemical and physicochemical techniques for concentrating and purifying viruses.

For practical reasons, high-titre virus is desirable, especially when a large number of cells are infected. In addition, high titres are a requirement for transduction of a large percentage of certain cell types. For example, the frequency of human hematopoietic progenitor cell infection is strongly dependent on vector titre, and useful frequencies of infection occur only with very high-titre stocks (Hock and Miller (1986) Nature 320: 275-277; Hogge and Humphries (1987) Blood 69: 611-617). In these cases, it is not sufficient simply to expose the cells to a larger volume of virus to compensate for a low virus titre. On the contrary, in some cases, the concentration of infectious vector virions may be critical to promote efficient transduction.

Several methods for concentration of retroviral vectors have been developed, including the use of centrifugation (Fekete and Cepko 1993 Mol Cell Biol 13: 2604-2613), hollow fibre filtration (Paul et al 1993 Hum Gene Ther 4; 609-615) and tangential flow filtration (Kotani et al 1994 Hum Gene Ther 5: 19-28). For example, retroviral vectors are concentrated with Macrosep centrifugal concentrators with a 300-kDa membrane cut-off (Pall Filtron, Glen Cove, N.Y.). The concentrators are spun at 3000×g for 90 min, reducing the 15-ml samples to 1.5 ml. Alternatively virus stocks are concentrated by centrifuging virus preparations at 6000×g for 16 hr at 4° C. (Bowles et al. (1996) Hum. Gene Ther. 7, 1735-1742).

Although a 20-fold increase in viral titre can be achieved using centrifugation, the relative fragility of retroviral Env protein limits the ability to concentrate retroviral vectors. While this problem can be overcome by substitution of the retroviral Env protein with the more stable VSV-G protein, which allows for more effective vector concentration with better yields, it suffers from the drawback that the VSV-G protein is quite toxic to cells.

Some alternative approaches to developing high titre vectors for gene delivery have included the use of: (i) defective viral vectors such as adenoviruses, adeno-associated virus (AAV), herpes viruses, and pox viruses and (ii) modified retroviral vector designs.

Thus, it is highly desirable to use high-titre virus preparations in both experimental and practical applications.

As used herein, the term “high titre” means an effective amount of a retroviral vector or particle, which is capable of transducing a target site—such as a cancer cell.

The term “effective amount” means an amount of a regulated retroviral vector or vector particle which is sufficient to induce expression of a therapeutic gene at a target site.

The present inventors have previously found that it is possible to get good in vitro transduction with the MLV 4070A envelope protein, but have now demonstrated that this level cannot be maintained when the concentration of envelope molecules, either particulate or free, exceeds the concentration of available receptors. Indeed, concentrating MLV 4070A pseudotyped vector preparations decreases, rather than increases, their transducing ability. The transducing power can be regained by diluting the concentrated stocks (Slingsby J et al. (2000) Hum. Gene Ther. 11, 1349).

The cognate receptor for the MLV 4070A envelope is a sodium dependent phosphate symporter, denoted Pit2 (Miller et al. (1994) PNAS 91, 78) that is expressed on the surface of a wide range of cells, making them targets for infection. However, as reported in the literature (Uckert et al (1998) Hum. Gene Ther. 9, 2619) and borne out by the present inventor's own work the level of Pit2 expression is low and can limit the maximum achievable transduction efficiencies.

The cognate receptor for the RD114 envelope is a sodium dependent neutral amino acid transporter, denoted ATB0 (or SLC1A5 or hASCT2) (Rasko et al. (1999) PNAS 96, 2129). This receptor is also expressed on a wide variety of cell types and at levels which exceed those for Pit2 (Tailor et al. (2000) J. Virol. 73, 4470).

The present inventors have found that unconcentrated RD114 pseudotyped vectors out-perform their MLV 4070A pseudotyped counterparts by approximately 3-fold on all cell types that we have tested. Surprisingly, this difference in performance is even more marked when using concentrated stocks, where we have found that RD114 pseudotyped vector preparations can be concentrated without compromising their transducing power. In fact after concentration, a boost in transduction efficiency is seen.

Preferably, a concentrated stock is at least 10⁸ particles/ml and 50% or more of target cells are transduced using a concentrated stock.

Packaging Cells

It is widely accepted that the low levels of gene transfer to target cells and tissues have compromised gene therapy trials and that results could be improved by delivering more vector particles to the patient.

Moreover, simple packaging cell lines, comprising a provirus in which the packaging signal has been deleted, have been found to lead to the rapid production of undesirable replication competent viruses through recombination. In order to improve safety, second generation cell lines have been produced wherein the 3′ LTR of the provirus is deleted. In such cells, two recombinations would be necessary to produce a wild type virus.

Packaging cell lines may be readily prepared (see also WO 92/05266), and utilised to create producer cell lines for the production of retroviral vector particles. As already mentioned, a summary of the available packaging lines is presented in “Retroviruses” (as above).

As used herein, the term “packaging cell” refers to a cell, which contains those elements necessary for production of infectious recombinant virus which are lacking in the RNA genome. Typically, such packaging cells contain one or more producer plasmids which are capable of expressing viral structural proteins (such as Gag, Pol and Env) but they do not contain a packaging signal.

Preferably, the retroviral vector system described herein is produced using human cells which has the dual advantage of minimising the generation of replication competent retroviruses (RCRs) and producing a vector that is relatively stable to the effects of human complement. Retroviral vectors have been produced in mouse cells and consequently the particles are associated with α-galactose sugar epitopes. Human serum contains an antibody to this molecule that results in inactivation of the vector via antibody-dependent complement-mediated lysis (Takeuchi et al. (1994) J. Virol. 68, 8001-8007).

Thus, the retroviral vector system of the present invention is preferably producing using a human retroviral packaging cell system. More preferably, the human retroviral packaging cell system is a FLY cell system. The FLY technology exploits a stringent selection system to ensure that the vector components are stably expressed at high levels. Using these cells, it is possible to produce retroviral vectors at significantly higher yields than previous methods (F L Cosset et al (1995) J Virol 69 7430-7436; Patience C et al (1998) J Virol 72, 2671).

To produce the FLY packing cells, suitable cell lines are used which include but are not limited to mammalian cells such as murine fibroblast derived cell lines or human cell lines. Alternatively, the packaging cell may be a cell derived from the individual to be treated such as a monocyte, macrophage, blood cell or fibroblast. The cell may be isolated from an individual and the packaging and vector components administered ex vivo followed by re-administration of the autologous packaging cells.

Preferably the packaging cell line is a human cell line such as HEK293, 293-T, TE671 and HT1080. More preferably, the packaging cell line is the human fibrosarcoma cell line HT1080 and the human rhabdomyosarcoma cell line TE671.

The engineered cells are able to trans-complement replication deficient viral genomes. The likelihood of producing RCRs in this type of system has been shown to be significantly reduced when the gag/pol and env functions are encoded on different stably integrated cassettes (Markowitz et al (1988) J Virol 62, 1120). This split function packaging arrangement has been used in FLY cells and as an additional safeguard, all the expression cassettes have been tailored to remove any overlapping sequences, thereby removing the chance of RCR production via homologous recombination.

To prepare the FLY cells, triple transfection is performed with three expression plasmids. One of the plasmids contains the MoMLV gag and pol genes, together with the blasticidin resistance gene. The other two plasmids each contain the phleomycin resistance gene with either the MoMLV 4070A amphotropic env gene or the feline endogenous retrovirus RD114 env gene. The expression of protein from all three plasmids is under the control of the FB29 Friend MLV LTR and in each case retroviral and resistance genes are encoded on the same mRNAs. The genes are arranged such that a 76 nucleotide or a 74 nucleotide spacer region separates the start codon of the downstream gene from the stop codon of the upstream gene. This spacing is sufficient to ensure that translation reinitiation is required before any selectable marker protein can be expressed. Translation reinitiation is an inherently inefficient process and it is this property that is exploited here to provide a novel and powerful selection methodology.

Vectors can be introduced into the FLY packaging cells either by transfection—such as using lipid formulations, calcium phosphate and electroporation—or by viral transduction. Preferably, vectors are introduced into the FLY packaging cells using viral transduction.

Since the FLY packing cells also express the RD114 envelope protein they are refractory to further infection by particles bearing the same RD114 envelope. Thus, the preferred method of delivery of the genome by viral transduction can only be achieved by using particles with a different pseudotype. In this case, a transient expression system can be used to prepare a vector stock pseudotyped with the VSV-G protein. These particles circumvent the RD114 infection block operating in the packaging cells and can deliver the vector genome with high efficiency.

In order to be absolutely certain that no VSV-G coding sequences are incorporated into the cell line, DNA can be extracted and analysed using various methods known in the art—such as PCR with primers specific to the VSV-G coding sequence.

Following viral transduction, genome-containing clones can be selected by growth in selection medium. Clones are then characterised with respect to vector identity and potency and the clone with the best profile is selected for use.

It is highly desirable to use high-titre virus preparations in both experimental and practical applications. A high-titre viral preparation for a producer/packaging cell is usually of the order of 10⁵ to 10⁷ t.u. per ml. (The titer is expressed in transducing units per ml (t.u./ml) as titred on a standard D17 cell line).

Producer Cell

A “producer cell” or “vector producing cell” refers to a cell, which contains all the elements necessary for production of recombinant viral vector particles and retroviral delivery systems.

There are two common procedures for generating producer cells. In one, the sequences encoding retroviral Gag, Pol and Env proteins are introduced into the cell and stably integrated into the cell genome; a stable cell line is produced which is referred to as the packaging cell line. The packaging cell line produces the proteins required for packaging retroviral RNA but it cannot bring about encapsidation due to the lack of a psi region. However, when a vector genome (having a psi region) is introduced into the packaging cell line, the helper proteins can package the psi-positive recombinant vector RNA to produce the recombinant virus stock. This can be used to transduce the therapeutic gene into recipient cells. The recombinant virus whose genome lacks all genes required to make viral proteins can infect only once and cannot propagate. Hence, the therapeutic gene is introduced into the host cell genome without the generation of potentially harmful retrovirus. A summary of the available packaging cell lines is presented in “Retroviruses” (1997 Cold Spring Harbour Laboratory Press Eds: J M Coffin, S M Hughes, H E Varmus pp 449).

The present invention also provides a packaging cell line comprising a viral vector genome, which is capable of producing a vector system of the present invention. For example, the packaging cell line may be transduced with a viral vector comprising the genome or transfected with a plasmid carrying a DNA construct capable of encoding the RNA genome.

The second approach is to introduce the three different DNA sequences that are required to produce a retroviral vector particle i.e. the env coding sequences, the gag-pol coding sequence and the defective retroviral genome containing one or more NOIs (nucleotides of interest) into the cell at the same time by transient transfection. This procedure is referred to as transient triple transfection (Landau & Littman 1992; Pear et al. 1993). The triple transfection procedure has been optimised (Soneoka et al. 1995; Finer et al. 1994). WO 94/29438 describes the production of producer cells in vitro using this multiple DNA transient transfection method. WO 97/27310 describes a set of DNA sequences for creating retroviral producer cells either in vivo or in vitro for re-implantation. Preferably, the packaging cells of the present invention are prepared by transient transfection. More preferably, transient transfection comprises introducing three vectors comprising the OB83 genome (pOB83), the MoMLV gag/pol gene (pHIT60) and the VSV-G env gene (pRV67) in to HEK 293 cells.

By using producer/packaging cell lines, it is possible to propagate and isolate quantities of retroviral vector particles (e.g. to prepare suitable titres of the retroviral vector particles) for subsequent transduction of, for example, a site of interest (such as the site of a tumour). Producer cell lines are usually better for large-scale production of vector particles.

Transient transfection has numerous advantages over the packaging cell method. In this regard, transient transfection avoids the longer time required to generate stable vector-producing cell lines and is used if the vector genome or retroviral packaging components are toxic to cells. If the vector genome encodes toxic genes or genes that interfere with the replication of the host cell, such as inhibitors of the cell cycle or genes that induce apoptosis, it may be difficult to generate stable vector-producing cell lines, but transient transfection can be used to produce the vector before the cells die. Also, cell lines have been developed using transient infection that produce vector titre levels that are comparable to the levels obtained from stable vector-producing cell lines (Pear et al. 1993, PNAS 90:8392-8396).

Producer cells/packaging cells can be of any suitable cell type. Producer cells are generally mammalian cells but can be, for example, insect cells.

Preferably the envelope protein sequences, and nucleocapsid sequences are all stably integrated in the producer and/or packaging cell. However, one or more of these sequences could also exist in episomal form and gene expression could occur from the episome.

Preferably, the producer cell is obtainable from a stable producer cell line. More preferably, the stable producer cell line is RD/83.

Preferably, the producer cell line is stable in culture for two months or more.

Therapeutic Gene

According to the present invention one or more therapeutic genes may be delivered to a target cell in vivo or in vitro.

As used herein the term “therapeutic gene” refers to a gene that is capable of eliciting a therapeutic or preventative effect or encodes a protein that is capable of eliciting a therapeutic or preventative effect.

The therapeutic gene may be any suitable nucleotide sequence, and need not necessarily be a complete naturally occurring DNA or RNA sequence that can be used in therapy. Thus, the therapeutic gene can be, for example, a synthetic RNA/DNA sequence, a recombinant RNA/DNA sequence (i.e. prepared by use of recombinant DNA techniques), a cDNA sequence or a partial genomic DNA sequence, including combinations thereof. The sequence need not be a coding region. If it is a coding region, it need not be an entire coding region. In addition, the RNA/DNA sequence can be in a sense orientation or in an anti-sense orientation. Preferably, it is in a sense orientation.

The therapeutic gene may be capable of blocking or inhibiting the expression of a gene in the target cell. For example, the therapeutic gene may be an antisense sequence. The inhibition of gene expression using antisense technology is well known.

The therapeutic gene or a sequence derived therefrom may be capable of “knocking out” the expression of a particular gene in the target cell. There are several “knock out” strategies known in the art. For example, the therapeutic gene may be capable of integrating in the genome of a target cell so as to disrupt expression of the particular gene. The therapeutic gene may disrupt expression by, for example, introducing a premature stop codon, by rendering the downstream coding sequence out of frame, or by affecting the capacity of the encoded protein to fold (thereby affecting its function).

Alternatively, the therapeutic gene may be capable of enhancing or inducing ectopic expression of a gene in the target cell. The therapeutic gene or a sequence derived therefrom may be capable of “knocking in” the expression of a particular gene.

Suitable therapeutic genes include but are not limited to: sequences encoding cytokines, chemokines, hormones, antibodies, anti-oxidant molecules, engineered immunoglobulin-like molecules, a single chain antibody, fusion proteins, enzymes, immune co-stimulatory molecules, immunomodulatory molecules, anti-sense RNA, a transdominant negative mutant of a target protein, a toxin, a conditional toxin, an antigen, a tumour suppresser protein and growth factors, membrane proteins, vasoactive proteins and peptides, anti-viral proteins and ribozymes, and derivatives thereof (such as with an associated reporter group) and pro-drug activating enzymes.

As used herein, “antibody” includes a whole immunoglobulin molecule or a part thereof or a bioisostere or a mimetic thereof or a derivative thereof or a combination thereof. Examples of a part thereof include: Fab, F(ab)′2, and Fv. Examples of a bioisostere include single chain Fv (ScFv) fragments, chimeric antibodies, bifunctional antibodies.

The term “mimetic” relates to any chemical, which may be a peptide, polypeptide, antibody or other organic chemical which has the same binding specificity as the antibody.

The term “derivative” as used herein includes chemical modification of an antibody. Illustrative of such modifications would be replacement of hydrogen by an alkyl, acyl, or amino group.

Preferably, the expression product(s) encoded by the therapeutic gene encodes a pro-drug activating enzyme. The principle of this therapy is to deliver a gene encoding an enzyme that transforms a non-toxic drug in to a toxic compound (Paillard et al. (1997) HGT 8, 1733-1735) and is referred to as “suicide gene therapy”. Cells that are expressing the suicide gene metabolise the drug and are killed. In practice the metabolite is not completely restricted to the tumour cell but provided that the toxic metabolite has some selectivity towards tumours, this is beneficial.

Various pro-drug activating enzymes are known in the art. The best-characterised enzyme/prodrug system uses the herpes simplex virus thymidine kinase enzyme that can specifically transform nucleoside analogues such as Aciclovir or Ganciclovir into monophosphorylated molecules. Cellular enzymes cannot perform this transformation. The monophosphates can however be converted to triphosphates that can be used in DNA synthesis but once incorporated into a DNA chain further elongation is blocked. This premature dispersed termination event leads to cell death. Several other prodrug-activating systems are also known—such as cytosine deaminase, which activates 5′ fluorocytosine to 5′ fluoruracil; E. coli nitroreductase, which activates CB1954 and cytochrome P450 2B6 which activates cyclophosphamide and ifosfamide.

Preferably, the therapeutic gene encodes the enzyme cytochrome P450 2B6 (GenBank accession no. M29874) and the pro-drugs are cyclophosphamide and/or ifosfamide. For example, cytochrome P450 2B6 converts the prodrug cyclophosphamide to the active phosphoramide mustard and acrolein. The phosphoramide mustard interacts with DNA to form cross-links. This has limited effects on quiescent cells but once the cell divides the cross-links result in DNA fragmentation and damage and cell death by apoptosis.

The expression product(s) encoded by the therapeutic gene may be proteins, which are secreted from the cell. Alternatively the expression product(s) encoded by the therapeutic gene are not secreted and are active within the cell. For some applications, it is preferred for the therapeutic gene expression product to demonstrate a bystander effect or a distant bystander effect; that is the production of the expression product in one cell leading to the modulation of additional, related cells, either neighbouring or distant (e.g. metastatic), which possess a common phenotype.

Pharmaceutical Compositions

The pharmaceutical compositions of the present invention comprise a therapeutically effective amount of the retroviral vector system.

The pharmaceutical compositions may be for human or animal usage in human and veterinary medicine and will typically comprise any one or more of a pharmaceutically acceptable diluent, carrier, or excipient. Preferably, the pharmaceutical compositions are for human usage in human medicine. Acceptable carriers or diluents for therapeutic use are well known in the pharmaceutical art, and are described, for example, in Remington's Pharmaceutical Sciences, Mack Publishing Co. (A. R. Gennaro edit. 1985). The choice of pharmaceutical carrier, excipient or diluent can be selected with regard to the intended route of administration and standard pharmaceutical practice. The pharmaceutical compositions may comprise as, or in addition to, the carrier, excipient or diluent any suitable binder(s), lubricant(s), suspending agent(s), coating agent(s), solubilising agent(s).

Preservatives, stabilizers, dyes and even flavoring agents may be provided in the pharmaceutical composition. Examples of preservatives include sodium benzoate, sorbic acid and esters of p-hydroxybenzoic acid. Antioxidants and suspending agents may be also used.

There may be different composition/formulation requirements dependent on the different delivery systems. By way of example, the pharmaceutical composition of the present invention may be formulated to be administered using a mini-pump or by a mucosal route, for example, as a nasal spray or aerosol for inhalation or ingestible solution, or parenterally in which the composition is formulated by an injectable form, for delivery, by, for example, an intravenous, intramuscular, intratumoral or subcutaneous route. Preferably, the pharmaceutical composition of the present invention is formulated to be administered parenterally in which the composition is formulated by an injectable form, for delivery, by, for example, an intratumoral route.

Alternatively, the formulation may be designed to be administered by a number of routes.

If the retroviral vector system is to be administered mucosally through the gastrointestinal mucosa, it should be able to remain stable during transit though the gastrointestinal tract; for example, it should be resistant to proteolytic degradation, stable at acid pH and resistant to the detergent effects of bile.

Where appropriate, the pharmaceutical compositions may be administered by inhalation, in the form of a suppository or pessary, topically in the form of a lotion, solution, cream, ointment or dusting powder, by use of a skin patch, orally in the form of tablets containing excipients such as starch or lactose, or in capsules or ovules either alone or in admixture with excipients, or in the form of elixirs, solutions or suspensions containing flavouring or colouring agents, or the pharmaceutical compositions can be injected parenterally, for example intravenously, intramuscularly or subcutaneously. For parenteral administration, the compositions may be best used in the form of a sterile aqueous solution, which may contain other substances, for example enough salts or monosaccharides to make the solution isotonic with blood. For buccal or sublingual administration the compositions may be administered in the form of tablets or lozenges which can be formulated in a conventional manner.

Administration

The retroviral vector system may be administered alone but will generally be administered as a pharmaceutical composition—e.g. when the components are is in admixture with a suitable pharmaceutical excipient, diluent or carrier selected with regard to the intended route of administration and standard pharmaceutical practice.

If the retroviral vector system encodes a pro-drug activating enzyme then the retroviral vector system will generally be administered in combination with a pro-drug. The retroviral vector system and the pro-drug may be administered at the same time, before or after administration of the retroviral vector system. For example, the pro-drug may be administered one week after the first administration of the retroviral vector system.

For example, the components can be administered in the form of tablets, capsules, ovules, elixirs, solutions or suspensions, which may contain flavouring or colouring agents, for immediate-, delayed-, modified-, sustained-, pulsed- or controlled-release applications.

If the pharmaceutical is a tablet, then the tablet may contain excipients—such as microcrystalline cellulose, lactose, sodium citrate, calcium carbonate, dibasic calcium phosphate and glycine, disintegrants such as starch (preferably corn, potato or tapioca starch), sodium starch glycollate, croscarmellose sodium and certain complex silicates, and granulation binders such as polyvinylpyrrolidone, hydroxypropylmethylcellulose (HPMC), hydroxypropylcellulose (HPC), sucrose, gelatin and acacia. Additionally, lubricating agents such as magnesium stearate, stearic acid, glyceryl behenate and talc may be included.

Solid compositions of a similar type may also be employed as fillers in gelatin capsules. Preferred excipients in this regard include lactose, starch, a cellulose, milk sugar or high molecular weight polyethylene glycols. For aqueous suspensions and/or elixirs, the agent may be combined with various sweetening or flavouring agents, colouring matter or dyes, with emulsifying and/or suspending agents and with diluents such as water, ethanol, propylene glycol and glycerin, and combinations thereof.

The routes for administration (delivery) may include, but are not limited to, one or more of oral (e.g. as a tablet, capsule, or as an ingestible solution), topical, mucosal (e.g. as a nasal spray or aerosol for inhalation), nasal, parenteral (e.g. by an injectable form), gastrointestinal, intraspinal, intraperitoneal, intramuscular, intravenous, intrauterine, intraocular, intradermal, intracranial, intratracheal, intratumoural, intravaginal, intracerebroventricular, intracerebral, subcutaneous, ophthalmic (including intravitreal or intracameral), transdermal, rectal, buccal, vaginal, epidural, sublingual or systemic.

For some embodiments, preferably, the route of administration is intratumoral. The injection site may be pre-treated with a local superficial injection of, for example, 2.0% lignocaine. The retroviral vector system described herein may be injected along multiple different tracks within the tumour nodule in order to obtain as wide a dispersion as possible.

Multiple administrations of the vector may give improved gene transfer. There is a rational expectation that this could be true for retroviral vectors because these are limited by the cell cycle status of the target cells. Repeated administrations allow cells in different stages of the cell cycle to be accessed by the vector at different times. Thus, for example, the retroviral vector may be administered in two treatments at each dosage level at a 24 hr interval.

Dose Levels

Typically, a physician will determine the actual dosage which will be most suitable for an individual subject. The specific dose level and frequency of dosage for any particular patient may be varied and will depend upon a variety of factors including the activity of the specific compound employed, the metabolic stability and length of action of that compound, the age, body weight, general health, sex, diet, mode and time of administration, rate of excretion, drug combination, the severity of the particular condition, and the individual undergoing therapy.

Each patient may be given an injection of an appropriate volume of retroviral vector system relative to the nodule size. For example, a 1 ml dose for use in tumours of 0.5 to 1.5 cm longest dimension; a 2 ml dose for tumours of 1.6 to 2.5 cm longest dimension; a 4 ml dose for tumours of greater than 2.5 cm longest dimension.

The volumes per tumour mass may be based upon an algorithm described by Stopeck et al (1997) J Clin Oncol 15, 341 for the administration of DNA based gene therapy. This study suggested the range of 1.0 ml per 0.5 cm to 1.0 cm of dimension with tumours greater than 3 cms receiving 4.0 ml.

For some embodiments, preferably, the maximum dose that will be used is for 5×10⁹ cells per 0.5 cm³. There are approximately 10⁹ cells per cm³ of tissue. Therefore this dose is approximately 10 fold higher than that required to treat all of the cells if the procedure is 100% effective.

Preferably a dose escalation protocol is followed. For example the vector system may be administered by intratumoral injection at escalating doses up to a maximum practical dose of 1×10⁹ lac2 transforming units (Ltu) per 0.5 cm diameter of tumour mass.

Formulation

The component(s) may be formulated into a pharmaceutical composition, such as by mixing with one or more of a suitable carrier, diluent or excipient, by using techniques that are known in the art.

Preferably, the retroviral vector system is administered in an aqueous formulation buffer comprising: Tris, NaCl, lactose, human serum albumin and protamine sulphate. More preferably, the retroviral vector system is administered in an aqueous formulation buffer comprising 19.75 mM Tris, 37.5 mM NaCl, 40 mg/ml lactose, 1 mg/ml human serum albumin and 5 μg/ml protamine sulphate pH 7.0. All the components used are PhEur or equivalent. Protamine sulphate and HSA are purchased as the licensed products Prosulf and Albutein respectively.

Target Cell

The retroviral vector system used in the present invention is particularly useful in delivering a therapeutic gene to a target cell—in particular a cancer cell. The cancer cell may be part of a solid tumour.

Thus, the retroviral vector system used in the present invention is useful in treating and/or preventing cancer.

In particular the retroviral vector system is useful in treating and/or preventing solid tumours for example, breast cancer, prostate cancer, ovarian cancer pancreatic cancer, head and neck cancer or melanoma. In a highly preferred embodiment the system is useful against breast cancer.

As used herein, the terms “treatment” “treating” and “therapy” include curative effects, alleviation effects, and prophylactic effects.

General Recombinant DNA Methodology Techniques

Although in general the techniques mentioned herein are well known in the art, reference may be made in particular to Sambrook et al., Molecular Cloning, A Laboratory Manual (1989) and Ausubel et al., Short Protocols in Molecular Biology (1999) 4th Ed, John Wiley & Sons, Inc. PCR is described in U.S. Pat. No. 4,683,195, U.S. Pat. No. 4,800,195 and U.S. Pat. No. 4,965,188.

The invention will now be further described by way of Examples, which are meant to serve to assist one of ordinary skill in the art in carrying out the invention and are not intended in any way to limit the scope of the invention.

### EXAMPLES

### Example 1

### Construction of OB80 and OB83

i. OB80

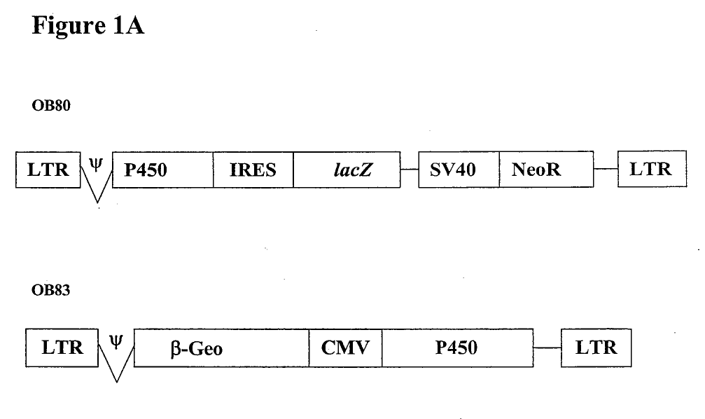

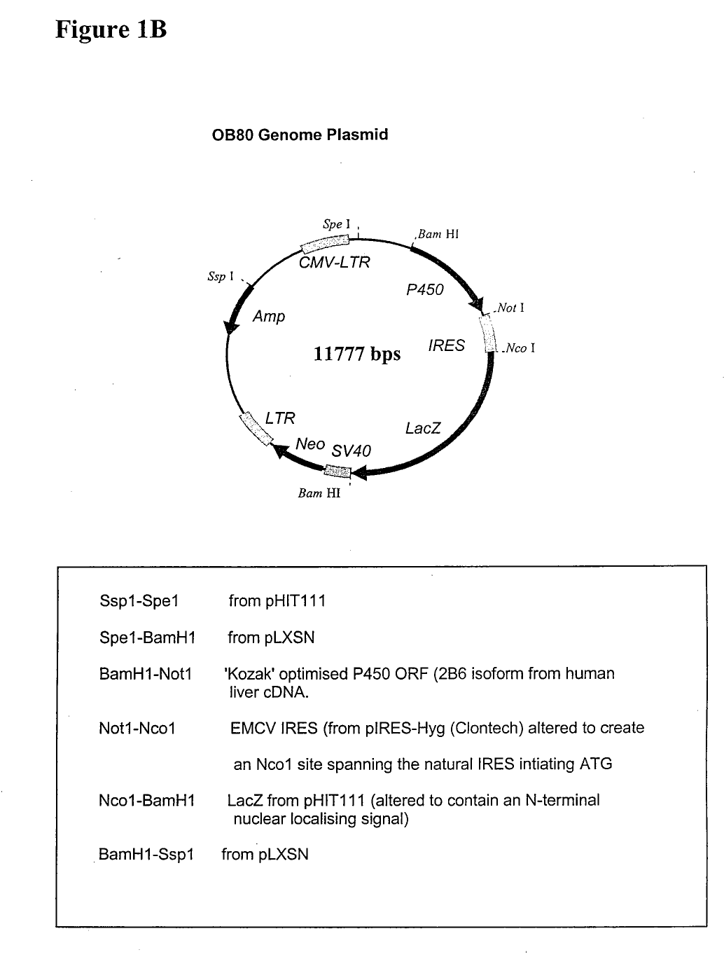

A diagrammatic representation of the vector genome of OB80 is shown in FIG. 1A and a map of the OB80 genome plasmid with a key to the derivation of its sequence shown in FIG. 1B.

The enzyme P450 2B6 is encoded by a single identified gene, designated CYP2B6 (Genbank Accession no M29874). First strand cDNA containing the coding region of CYP2B6 is obtained by reverse transcription from commercial liver total RNA (Clontech) using an oligo-dT primer. The corresponding double-stranded cDNA fragment is amplified by PCR using the primers P450F (sequence: CAG ACC ATG GAA CTC AGC GT) and P450R (sequence: GGA CAC TGA ATG ACC CTG GA). Due to the low abundance of CYP2B6 mRNA in the liver RNA preparation, a second round of PCR is performed on the first PCR product using oligonucleotide primers P450FOR (sequence: TCA TGC TAG CGG ATC CAC CAT GGA ACT CAG CGT C) and P450REV (sequence: AAA ATC ACA CTC TAG ATT CCC TCA GCC CCT TCA GC) for the plus and minus strands respectively. The final PCR product contains a NheI site, a BamHI site and a consensus translation initiation signal that is added upstream of the 5′-end of the coding region of CYP2B6, as well as an XbaI site after the 3′ end of the open reading frame.

The cDNA fragment generated from the PCR amplification is cloned into the pCRII-TOPO vector using the TOPO TA Cloning Kit from Invitrogen. A resulting plasmid clone contains the sequence of CYP2B6 as determined by Yamano et al. (1989), Biochemistry 28:7340, except for a single base change at nucleotide 1201. The sequence of the complete CYP2B6 insert is independently confirmed on two separate occasions, once using manual sequencing and once using an ABI automated DNA sequencer. The single base change is presumed to have been introduced as an error in the PCR amplification since several other clones sequenced showed the published sequence at this position. The single base difference does not affect the sequence of the protein translated from this clone and hence the open reading frame of OBM27 encodes wild-type human P450 2B6.

A plasmid, pLNSX, containing the MLV LTR is digested with Nhe1 and the fragment containing the plasmid backbone and LTR sequences is re-ligated to produce pMLVLTR. The MLV transcription enhancer is removed from pMLVLTR by digestion with Nhe1 and Xba1 and replaced by a synthetic oligonucleotide representing 3 copies of the hypoxia response element (HRE) from a mouse PGK gene to form plasmid pHRE-LTR.

A retroviral vector MOI is obtained from Sunyoung Kim, Seoul National University, South Korea. An internal Nhe1 fragment containing the sequences between the LTRs is isolated from this vector and introduced at the Nhe1 site of the plasmid pHRE-LTR to form pMOI-HRE1. The E. coli gene lacZ, which encodes β-galactosidase is cloned using PCR from pSP72 (Invitrogen) and the N-terminus is simultaneously modified to contain a nuclear localisation signal for mammalian cells from the SV40 T-antigen using the following oligonucleotide primers.

Lead primer
CTC AGC ACC CTC GAG AGG CCT GCC ACC ATG GGG ACT
GCT CCA AAG AAG AAG CGT AAG GTA GTC GTT TTA CAA
CGT CGT GAC
Complement
GAT CGG TGC GGG CCT CTT CG

The sequence of the constructed gene is confirmed by DNA sequencing. The Nls-lacZ coding sequence is isolated from pSP72 as a Stu1/Sal1 fragment and introduced into the Stu1/Xho1 of pMOI-HRE creating pMOI-Z-HRE.

To remove a false ATG start and other restriction sites the CYP2B6 plasmid is cut with Spe1 and Nhe1 then religated. The P450 coding sequence is isolated as a BamH1/Xho1 fragment and introduced into the BamH1/Sal1 site of pMOI-Z-HRE creating pMOI-P450-Z-HRE

A neomycin phosphotransferase expression cassette (TK promoter-neo-TK polyA) is isolated from the plasmid Selectavector-Neo (Ingenius) as an EcoRV fragment and cloned into the unique Bst 1107 site of pHRE-LTR generating pHRE-LTR-neo.

The P450 IRES LacZ cassette is isolated from pMOI-P450-Z-HRE as an Nhe1 fragment and cloned into the Nhe1 site of pHRE-LTR-neo to create pMOI-P450-Z-HRE-neo.

The 5′ LTR is replaced with a CMV promoter as follows. The Pvu1-Spe1 fragment containing the CMV/R/U5 fragment from pHIT111 (Soneoka et al 1995 Nucleic Acids Res 23, 628) is cloned into the Pvu1/Xba1 sites of pSP72 to make pSP72-CMV-HIT. Bal1/Sap1 including the IRES-lacZ is isolated from pMOI-Z-HRE and cloned into the Bal1/Sap1 site of pSP72-CMV-HIT to create pCMV-Z-HRE.

Sal1/BglII fragment containing the CMV-LTR is isolated from pCMV-Z-HRE and cloned into the Xho1/BglII of pSP72 to create the plasmid pSP72-CMV-MOI.

Finally, the Sca1/BglII fragment from pSP72-CMV-MOI is used to replace the equivalent fragment of pMOI-P450-Z-HRE-neo to create the plasmid pCMV-genome-HRE-neo (OB72).

When the vector pCMV-genome-HRE-neo is tested in transient virus production experiments, the titres obtained are lower than expected. As a result, the following additional changes are made.

1. Exchanging the FMDV IRES for the EMCV IRES to Enhance β-Galactosidase Expression Levels:

The EcorV fragment containing a p450-IRES-lacZ fragment is cloned into the pSP72 to make pSP72-FMDV. A PCR reaction is carried out using the Clontech plasmid pIRES-Hyg as a template with the following primers:

VSAT 79
(Not1 site in bold)
5′-CATGCATCTAGGGCGGCCGCACTAGAG-3′
VSAT81
(Nco1 site in bold)
5′-GGTTGTGGCCCATGGTATCATCGTGTTTTTCAAAGG-3′

The resulting PCR product contains the EMCV IRES DNA fragment with a Not1 site at the 5′-end and an Nco1 site spanning the natural EMCV IRES ATG initiation of translation. This product is cut with Not1 and Nco1 and cloned into the Not1-Nco1 digested pSP-FMDV thus replacing the FMDV IRES with the EMCV IRES such that the Nco1 site spans the lacZ ATG start site. This resulting plasmid is called pSP-EMCV-D4. Next, the EcoRV fragment from this vector (containing the p450-EMCVIRES-lacZ fragment) is cloned into EcoRV digested OB72 with the equivalent p450-FMDV-lacZ fragment removed. The resulting plasmid is named E1 and is equivalent to OB72 but with the FMDV IRES replaced by the EMCV IRES.

2. Changing Vector Backbone to Enhance Titres.

First the Ssp1-Spe1 fragment from CMV-R-U5 fragment is taken from pHIT111 (Soneoka et al 1995) and cloned into the Ssp1-Spe1 digested pLXSN (Miller et al 1990 Mol Cell Biol 10, 4239), thus replacing the U3 based LTR with the CMV equivalent. The resulting vector is called pRV583. In to the BamH1 site of this vector was next cloned the P450-EMCVIRES-lacZ BamH1 fragment from E1. The resulting vector is OB80.

ii. OB83

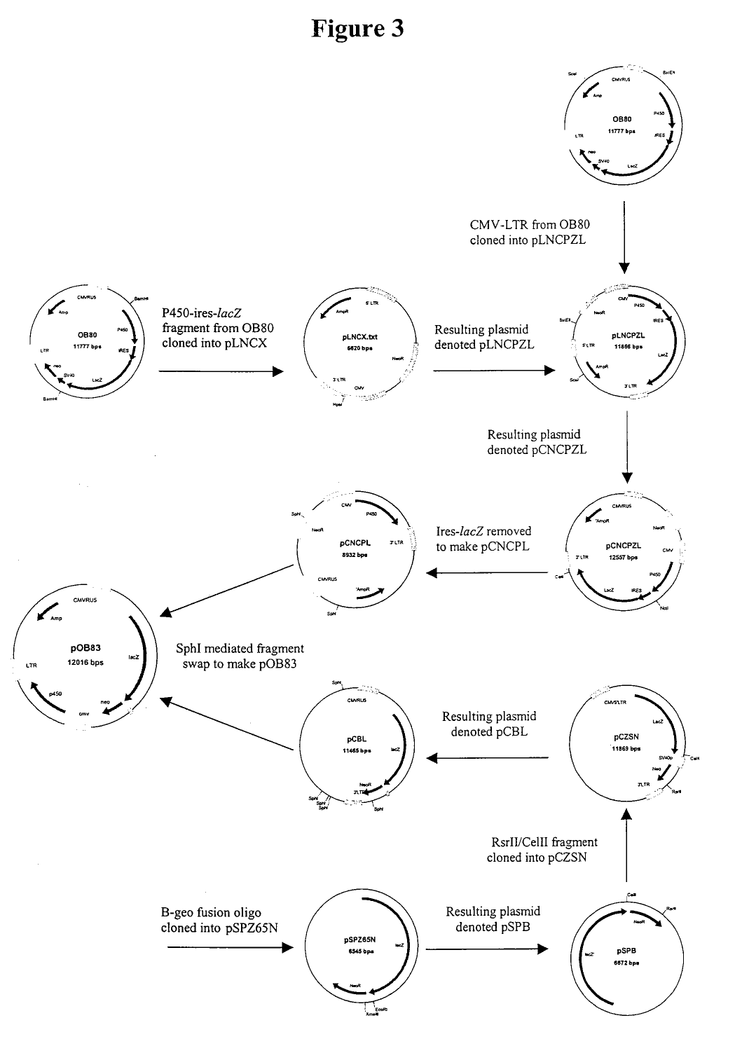

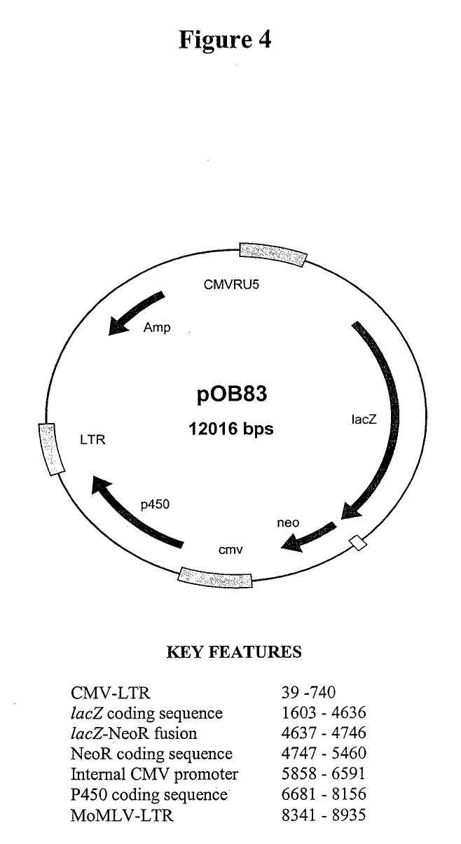

For ease of understanding, a diagrammatic representation of the vector genomes of OB83 is shown in FIG. 1A, complete cloning strategy is shown diagrammatically in FIG. 3 and a key features map is shown in FIG. 4.

The CYP2B6 gene is obtained by PCR amplification from human hepatocyte derived mRNA. The correct gene sequence is confirmed by comparison to the established sequence (GenBank accession no. M29874) prior to using the cDNA in any of the experiments described herein.

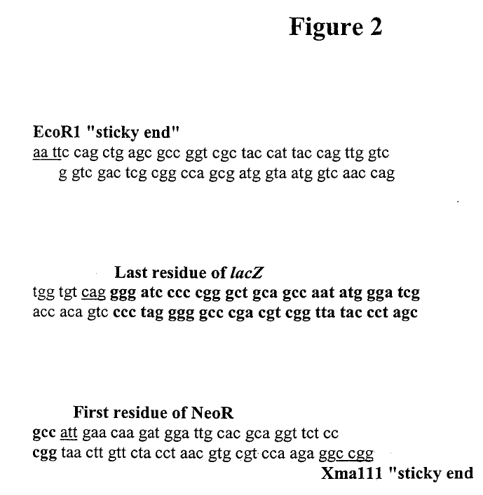

The P450-IRES-lacZ containing fragment is cut from plasmid pOB80 with BamH1 and its ends are blunted using T4 DNA polymerase. This fragment is then blunt-end ligated into the plasmid pLNCX (Miller A D et al. (1989) Biotechniques 7, p 980-990) cut with the enzyme Hpa1. The resulting construct was designated pLNCPZL. The 5′LTR of pLNCPZL is then substituted by the hybrid CMV-LTR CMV-R-U5) from the plasmid pOB80 by a Sca1/BstE11 mediated fragment swap. The resulting construct is designated pCNCPZL. In order to remove the IRES-lacZ from pCNCPZL it is cut with Not1 and Cel11 and the ends blunted using T4 DNA polymerase and then religated. The resulting construct is designated pCNCPL. To create the β-Geo fusion, plasmid pSPZ65N is cut with the enzymes EcoR1 and Xma111, removing a small (˜160 bp) fragment. This section is replaced with a piece of synthetic DNA designed from a published sequence (Friedrich & Soriano (1991) Genes Dev 5, 1513; Abram et al. (1997) Gene 196, 187) as shown in FIG. 2. The intermediate plasmid is designated pSPB. Sequence analysis is used to confirm that the fusion oligo had been inserted correctly. The newly created β-Geo sequence is cut from pSPB with Rsr11 and Cel11 and inserted into the plasmid pCZSN that is cut with the same enzymes. The intermediate plasmid is designated pCBL. This shuttling procedure is performed in preparation for the final cloning step. The final step is an SphI mediated fragment swap between pCBL and pCNCPL. The resulting construct (pCBCPL) is given the code OB83. At this point the identity of the complete genome is confirmed by cGLP sequence analysis (Lark Technologies). This analysis revealed 100% identity with the predicted sequence.

### Example 2

### Preparation of Fly Packaging Cell Lines

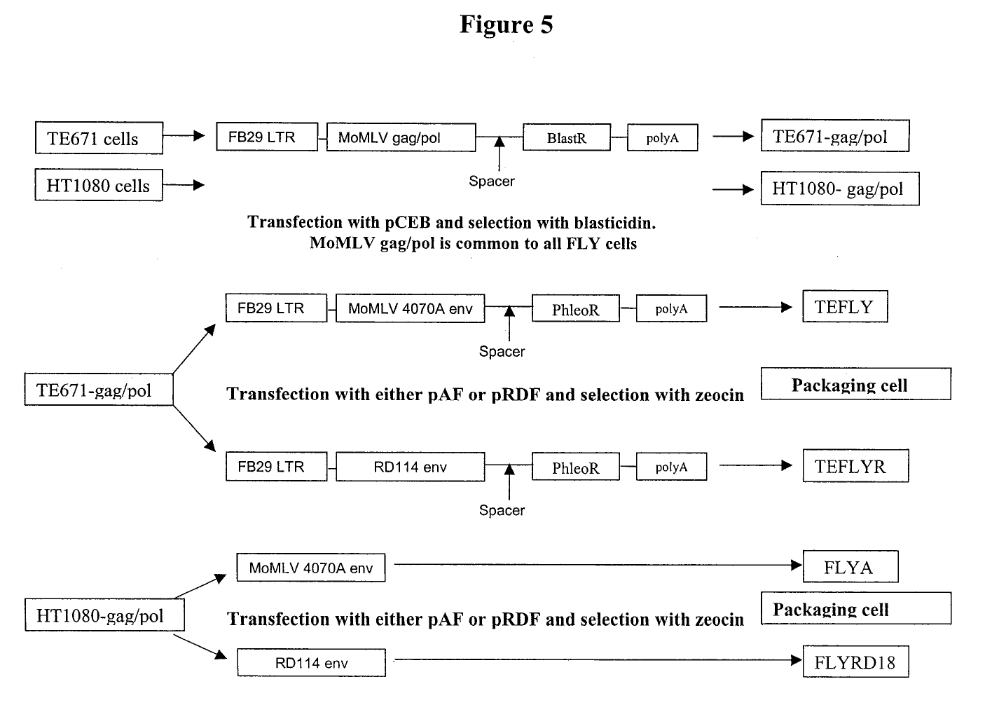

Diagrams of the individual expression cassettes and the derivation of the various FLY cell lines are shown in FIG. 5.

A virus stock containing the OB83 (or OB80) genome is made in a transient expression system as described by Soneoka et al. (1995) Nucleic Acids Res. 23, 628-633, using human 293 cells. The expression plasmid pRV67 (Kim et al. (1998) J. Virol. 72, 811-816) is used to pseudotype retroviral stocks with the VSV-G envelope protein. The retroviral genome is introduced into the packaging cell lines by retroviral transduction in the presence of 8 μg ml-1 Polybrene. VSV-G pseudotyped retrovirus is added to 50% confluent packaging cells at a low multiplicity of infection in 12-well plates. After 24 hr, the cells are split into 15 cm plates and 1 mg ml-1 G418 is added to select for expression of the neo gene, transcribed from within the OB83 genome. After 14 days, high titer producer cell lines are identified by end-point titration.

The FLY family of retroviral packaging cell lines have been described in F L Cosset et al (1995) J. Virol. 69, p 7430-7436. To prepare the FLY cells, three expression plasmids are produced. One of them (pCEB) contains the MoMLV gag and pol genes, together with the blasticidin resistance gene. The other two (pAF and pRDF) each contain the phleomycin resistance gene with either the MoMLV 4070A amphotropic envelope gene or the cat endogenous retrovirus RD114 envelope gene respectively. The expression of protein from all three constructs is under the control of the FB29 Friend MLV LTR and in each case retroviral and resistance genes are encoded on the same mRNAs. The genes are arranged such that a 76 nt (pAF and pRDF) or a 74 nt (pCEB) spacer region separates the start codon of the downstream gene from the stop codon of the upstream gene. This spacing is sufficient to ensure that translation reinitiation is required before any selectable marker protein can be expressed. Translation reinitiation is an inherently inefficient process and it is this property that is exploited here to provide a novel and powerful selection methodology.

The VSV-G pseudotyped OB83 viral particles are introduced into the TEFLYRD packaging cell using viral transduction. Once the OB83 genome has been delivered to the packaging cells in this way, they will begin to secrete vector particles bearing the RD114 envelope and the VSV-G envelope plays no further part in the process.

### Example 3

### Generation of the Producer Cell Line RD/83 (pOB83+TEFLYRD)

A process flow chart detailing the derivation of the RD/83 producer cell line is shown in FIG. 7.

Following transduction into TEFLYRD cells, OB83 genome-containing clones are selected by growth in G418-containing medium. These clones are characterised with respect to vector identity and potency to identify the clone with the best profile, denoted RD/83 (which produces virus containing the OB83 genome and the RD114 envelope).

In order to be absolutely certain that no VSV-G coding sequences are incorporated into the RD/83 cell line, DNA is extracted and subjected to PCR analysis using primers specific to the VSV-G coding sequence. The assay which has a demonstrated absolute sensitivity of between 13 and 130 copies failed to detect any VSV-G sequences in 100 ng of DNA (˜2500 cell equivalents). Therefore, it is concluded that RD/83 does not contain any VSV-G coding sequences. These data are shown in FIG. 6.

### Example 4

### Assessment of the Genetic Stability of RD/83

RD/83 cells are serially passaged in a 3-day, 4-day rotation both in the presence and absence of the selective agent G418, for an extended period of three months. Samples of medium are taken throughout the culture period and assessed by titration for vector yield. Typical values obtained (Ltu/ml) are tabulated below and it can be seen that all of the values obtained are within 0.5 log units of each other and as such, vector production remains stable over three months. The fact that equal values are obtained with and without G418 selection indicates that there has not been any promoter shutdown of the genome LTR in the absence of selection.

| | Time 0 | 1 month | 2 month | 3 month |
|---|---|---|---|---|
| −G418 | 6.8 × 106 | 4.1 × 106 | 3.4 × 106 | 4.05 × 106 |
| +G418 | | 3.3 × 106 | 1.7 × 106 | 4.2 × 106 |

By Western blot analysis, the levels of protein expression from the three cassettes in RD/83 cells (gag, env and β-gal) are determined. These data are presented in FIG. 14. As predicted from the results of the vector yield titration experiment, it can be seen that the level of expression from all three cassettes is unchanged after 3 months in culture.

### Example 5

### Gene Transfer Efficiency of OB80/4070A, OB83/4070A and OB83/RD114 in In Vitro Cell Cultures

The transducing power of three two-fold dilution series of concentrated OB80 and OB83 retroviral vector stocks is determined by transducing HT1080 target cells and then estimating the number of transduced cells by X-Gal staining.

This assay uses the β-galactosidase cleavage of a substrate, X-Gal, that produces an insoluble indigo precipitate in the presence of ferrous ions. Briefly, cells are fixed in 4% paraformaldehyde (in PBS+2 mM MgCl2) for 5 minutes. The fixed cells are washed in PBS before being covered with X-Gal staining solution (5 mM potassium ferricyanide, 5 mM potassium ferrocyanide, 2 mM magnesium chloride, 1 mg/ml X-Gal, in PBS pH 7.4). The cells are incubated for 5-24 hours (until optimal staining is achieved) at 37° C. in a humidified incubator. The cells are then examined microscopically and the number of blue cells as a percentage of the total is determined.

The immunohistochemical detection of cytochrome P450 is performed in an analogous way to that for β-galactosidase as described above using the appropriate primary and secondary detecting antibodies.

The results are presented in FIG. 8.

Transduction of HT1080 cells by concentrated OB83/RD114 shows that the number of cells transduced in the neat and two-fold diluted wells is >50% and is clearly visible to the naked eye, characterised by strong non-ires mediated expression of lacZ (B-geo). The level of transduction decreases with increasing dilution (Panel 1).

Transduction of HT1080 cells by OB80/4070A, is not visible in this image (Panel 2). Microscopic examination of the wells showed that the peak level of transduction occurred when the material was diluted four-fold. At this point the number of cells transduced was ˜20% and they showed characteristically weak ires mediated expression of lacZ.

Panel 3 shows the results obtained with the “hybrid vector” OB83/4070A, which has the OB83 genome and the 4070A pseudotype. The higher lacZ expression level obtained from this genome allows the transduced cells to be seen in this image. As with OB80/4070A, the peak level of transduction (˜20%) was seen at a four-fold dilution and after this transduction levels decreased with increased dilution

It is thus demonstrated that OB83 is better at transducing tumour cells than OB80 and, that transduction with vectors pseudotyped with RD114 is better than transduction with vectors pseudotyped with 4070A.

### Example 6

### Determination of the Level of Expression of P450 from OB80 and OB83

Having established that OB83 transduces cells with high efficiency, the level of P450 expression in the transduced cells is determined.

Preparations of OB80 and OB83 pseudotyped vector particles were made and then used to transduce naïve HT1080 target cells. To ensure that both preparations had performed similarly, a representative sample of the transduced cells was stained to detect β-galactosidase expression. For any subsequent analysis of these mixed populations of cells to be valid, the level of transduction achieved by both viruses must not only be similar, but also be high enough to ensure that positional integration effects do not skew the results. In the event, an acceptable level of transduction of >30% was achieved in both cases and DNA and RNA samples were extracted from the cells.

In a Northern blot analysis an equal amount (2 μg) of total RNA from both cell populations was immobilised on a nylon membrane and then probed with a radiolabelled probe specific for P450 sequences. The radioactive signals were then visualised by autoradiography and the resulting autoradiograph is shown in FIG. 9. A single transcript corresponding to the full-length genome (˜8000 bases) was detected in the OB80 transduced cells. Two transcripts (˜8300 bases and ˜2250 bases) corresponding to the full-length genome and the internal CMV controlled cassette, respectively were detected in OB83 transduced cells. From the autoradiograph, it can be seen that the expression level of P450 from the internal CMV promoter (˜2250 bases) in OB83 was greater than that from the LTR in both OB80 and OB83 (˜8000 bases and ˜8300 bases, respectively). The relative expression levels are measured on a phosphorimager and it is determined that the CMV promoter is about 4-fold stronger than the LTR.

Thus, it is demonstrated that once integrated, the level of expression of P450 from the internal CMV promoter of the OB83 genome is greater than the level of expression of P450 from OB80.

### Example 7

### The In Vitro Potency of OB83/RD114

The cell proliferation ELISA is used to assess the in vitro potency of OB83/RD114 in T47D and MDA231 breast cancer cell lines and LNCap and PC3 prostate cancer cell lines.

The cell proliferation assay kit is obtained as a quality controlled assay kit from Boehringer (Cat no. 1 669 915). Briefly, the cell proliferation ELISA is based on the incorporation of 5-bromo-2′-deoxy-uridine (BrdU) into the genome of proliferating cells. Cells are plated and cultured in the presence of BrdU. During the labelling period BrdU is incorporated in preference to thymidine into the DNA of cycling cells. The labelling medium is removed, the cells fixed and DNA denatured. The BrdU incorporated is detected by an anti-BrdU POD antibody that produces light in the presence of the substrate. The reaction product is quantified using a scanning multi-well luminometer.

Both cyclophosphamide and ifosfamide have been used in these assays. The data from the breast cancer lines are shown graphically in FIG. 10 and those from the prostate lines are shown in FIGS. 11 and 12.

These data show that OB83 mediates a marked inhibition of cell proliferation in the cell lines tested and that the effect is seen both with cyclophosphamide and ifosfamide.

### Example 8

### Comparison of Gene Transfer Efficiency of OB80 and OB83 In Vivo after Intratumoral Injection

To establish whether the in vitro gene transfer capability of OB83 is extended to an in vivo scenario, MDA231 and LNCaP prostate carcinoma cell line human tumour xenografts were established in nude mice and then injected with OB83/RD114.

The nude mice used were the BALB/cOlaHsd-nu/nu mouse. Two different human tumour xenografts were used, one breast cancer cell line MDA231 and the LNCaP prostate carcinoma cell line. These were selected on the basis of tissue origin as well as their ability to establish xenografts. Tumours were established sub-cutaneously and left to develop until a size of 50-80 mm³ is achieved. The time for establishment varies with each cell line. Two tumours per animal were established, one on each flank.

Four groups of tumour bearing mice were used for each xenograft being studied and for each vector. Three groups received OB83/RD114 at three different strengths (100×, 10× and 1×) and the fourth group receives a formulation buffer control injection. Three doses of 100 μl of the appropriate test article are administered by the intratumoural route on day 0, day 1 and day 2.

On day 5 and day 6 the animals in each group were injected intraperitoneally with either cyclophosphamide (4.4 mg), ifosfamide (7.32 mg) or formulation buffer. In this way a matrix of all possible combinations of test articles and prodrugs plus the relevant controls are established.

Tumours and tissues (lungs, liver spleen, ovaries, heart and brain) were harvested from two animals in each group at day 5 and from two further animals in each group on day 6. Tumours and tissues were harvested from all surviving mice when the tumours in the control group reach a maximum volume of 2000 mm³.

The level of gene transfer was assessed histologically. Frozen tumour samples (preclinical and clinical) were mounted in a cryomount OCT and then sectioned using a cryostat. The sequential 5 μm sections were mounted on slides, usually three sections per slide and stored frozen. Representative sections were then probed for the presence of products of the therapeutic gene (P450 2B6) and for the marker gene (lacZ). Expression of the marker gene was determined histologically using two approaches. In the first instance, the assessment for β-galactosidase expression in the frozen section was by X-gal histochemistry. This assay uses the β-galactosidase cleavage of a substrate, X-gal, to produce an insoluble indigo precipitate in the presence of ferrous ions. Frozen sections fixed in 2% paraformaldehyde (in PBS+2 mM MgCl₂) for 10 mins. Sections were then rinsed in PBS prior to transfer to X-gal staining solution (5 mM potassium ferricyanide, 5 mM potassium ferrocyamide, 2 mM magnesium chloride, 1 mg/ml X-gal, in PBS pH 7.4). Sections were incubated for 2-3 h at 37° C. or until optimal staining was achieved. If required a light counterstain was applied (30 secs OrangeG, 1% in 2% tungsto-phosphoric acid) prior to dehydration through an alcohol gradient and mounting in a proprietary mountant. If confirmatory data were required, the lacZ gene product can also be detected by immunohistochemical methods. Briefly, sections were fixed, desiccated and then incubated with a primary anti β-galactosidase antibody (rabbit polyclonal 5′-3′) diluted typically to 1/500 in serum supplemented tissue culture medium. Incubation was for 2-3 h at 37° C. or overnight at 4° C. Unbound primary antibody was rinsed off in PBS/0.05% Tween 20 prior to addition of secondary detection reagents. The Vectastain Elite ABC system was used according to manufacturers protocol to optimize sensitivity.

The immunohistochemical detection of cytochrome P450 2B6 was performed in an analogous way to that for β-galactosidase as described above using the appropriate primary and secondary detecting antibodies.

In order to establish whether the in vitro gene transfer capability of OB80 is extended to an in vivo scenario. LNCaP prostate carcinoma cell line human tumour xenografts were established in nude mice and then injected with OB80/4070A following a similar protocol to that described supra for OB80/RD114.

The results, which are shown in FIG. 13 show that, as with in vitro gene transfer, OB83 also outperforms OB80 in in vivo gene transfer in to the MDA231 tumour xenograft model. The same high levels of gene transfer were also seen in MDA468 tumour xenografts (data not shown).

### Example 9

### The In Vivo Potency of OB80/4070A—Clinical Data

OB80/4070A has been tested in a Phase I/II clinical trial. As well as the obvious safety aspects, the trial objectives were to assess gene transfer and efficiency of gene expression.

12 patients were recruited to the trial and administered with vector at 1×, 10× or 100× dose (1× dose=8×10e5) by direct intratumoural injection followed 1 week later by two courses of cyclophosphamide treatment, in which cyclophosphamide (100 mg/m²) was administered daily for 14 days followed by 14 days of no treatment and a further 14 days of cyclophosphamide treatment. The drug was well tolerated and no significant adverse signs of toxicity were observed. Gene transfer was detected in 10/12 patients. Two patients showed a partial response to treatment.

FIG. 15 illustrates patient lesions at time points 1 week and 12 weeks for patient BC1-104 and at week 0 and week 12 for patient BC1-101 after treatment Patient 101 received a 1× dose (8×10e5) and patient 104, a 10× dose (8×10e6) into each lesion. In both patients, significant regression of the injected lesions was observed.

Since the potential gene transfer and gene expression is predicted to be significantly improved with the OB83 vector, an enhancement of the effect would be predicted when this is used.

All publications mentioned in the specification are herein incorporated by reference. Various modifications and variations of the described methods and systems of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes of carrying out the invention which are obvious to those skilled in molecular biology or related fields are intended to be within the scope of the following claims.

1. A method for treating a solid tumor in a subject, comprising administering to a cancer cell in the subject an RD114 pseudotyped retroviral vector comprising a nucleotide sequence encoding cytochrome p450, wherein cytochrome p450 is expressed in the cancer cell; and administering to the subject cyclophosphamide or ifosfamide to activate the cytochrome p450 in the cancer cell, thereby inducing death of the cancer cell and treating the solid tumor in the subject.

2. The method according to claim 1, wherein the RD114 pseudotyped retroviral vector is an MLV-based retroviral vector.

3. The method according to claim 1, wherein the cytochrome p450 is cytochrome p450 2B6.

4. The method according to claim 1, wherein the cancer cell is selected from the group consisting of a breast cancer cell, a prostate cancer cell, an ovarian cancer cell, a pancreatic cancer cell, a head and neck cancer cell, and a melanoma cancer cell.

5. The method according to claim 1, wherein the RD114 pseudotyped retroviral vector is administered via an intratumoral route.

6. The method according to claim 1, wherein the RD114 pseudotyped retroviral vector is administered via an intravenous route.

7. The method according to claim 1, wherein the nucleotide sequence encoding cytochrome p450 is in operable linkage with a promoter.

8. The method according to claim 7, wherein the promoter is a cytomegalovirus (CMV) promoter.

9. The method according to claim 7, wherein the promoter is a hypoxia response element (HRE).
